(19) United States
(12) Patent Application Publication
(10) Pub. No.: US 2010/0261662 A1
(43) Pub. Date: Oct. 14, 2010

(54) UTILIZATION OF MURAL THROMBUS FOR LOCAL DRUG DELIVERY INTO VASCULAR TISSUE

(75) Inventors: Stefan G. Schreck, Irvine, CA (US); Charles S. Bankert, Irvine, CA (US); Kemal Schankereli, Irvine, CA (US)

Correspondence Address: KNOBBE MARTENS OLSON & BEAR LLP, 2040 MAIN STREET, FOURTEENTH FLOOR, IRVINE, CA, 92614, US

(73) Assignee: ENDOLOGIX, INC., Irvine, CA (US)

(21) Appl. No.: 12/757,900

(22) Filed: Apr. 9, 2010

Related U.S. Application Data
(60) Provisional application No. 61/168,199, filed on Apr. 9, 2009.

(51) Int. Cl. A61K 31/7032; A61M 1/00; A61K 9/00; A61K 31/353; A61P 9/10; A61P 29/00; A61P 7/02

(52) U.S. Cl. 514/25; 604/27; 424/484; 514/456

(57) ABSTRACT

The present disclosure is directed to methods and systems for stabilizing an extracellular matrix in a wall of a blood vessel. The method comprises delivering a therapeutic agent into mural thrombus, which covers the wall of the blood vessel. The agent is transported from the mural thrombus into the extracellular matrix of the vessel wall by diffusion. The agent then acts to reduce the enzymatic degradation of protein in the extracellular matrix.

### CROSS-REFERENCE TO RELATED APPLICATIONS

This application claims the benefit of U.S. Provisional Patent Application No. 61/168,199, filed on Apr. 9, 2009, the entire content of which is hereby incorporated by reference and should be considered part of this specification.

This application is related to U.S. Provisional Patent Application No. 60/987,261, filed Nov. 12, 2007, U.S. Provisional Patent Application No. 61/012,356, filed Dec. 7, 2007, U.S. Provisional Patent Application No. 61/127,654, filed May 14, 2008, U.S. Provisional Patent Application No. 60/987,268, filed Nov. 12, 2007, U.S. Provisional Patent Application No. 61/012,579, filed Dec. 10, 2007, U.S. Provisional Patent Application No. 60/533,443, filed on Dec. 31, 2003, and U.S. patent application Ser. No. 12/269,677, filed Nov. 12, 2008, which are each hereby incorporated by reference in their entireties as if fully set forth herein.

### BACKGROUND OF THE DISCLOSURE

1. Field of the Disclosure

The present disclosure relates to methods and agents for in-situ stabilization of vascular tissue.

2. Background and Summary of the Disclosure

Aortic aneurysm and aortic dissections involve the tissue of the aortic vessel. Over-expression of enzymes (matrix metalloproteinase) can break down the elastin and collagen structure in the wall. The vessel wall can become weak, dissect, and expand radially and axially in response to blood pressure. Degradation of the collagen structure can ultimately lead to aortic rupture and potential patient death.

Current surgical treatments for aortic aneurysms and dissections include the replacement of the diseased blood vessel with a vascular graft and endovascular placement of a stent graft to protect the weakened portion of the blood vessel from the pressure forces of the blood.

Pharmacological approaches for the treatment of aortic aneurysm are currently researched that are less invasive than the surgical repair. Agents considered for pharmacological interventions either target the inflammatory processes or enzymes responsible for the break down of elastin and collagen in the tissue. Agents may be chosen from anti-inflammatory drugs and matrix metalloproteinase (MMP) inhibitors, specifically MMP-2 and MMP-9. Examples of potential agents include statins and doxycycline.

Alternatively, the elastin and collagen may be stabilized against enzymatic degradation by cross-linking of the proteins. Rao et al. (Indian Journal of Biochemistry & Biophysics, vol. 81, June 1981) are believed to be the first to report in situ cross-linking of collagen using bioflavenoids. Rao et al. demonstrated that skin collagen in rats treated with catechin was stable against enzymatic degradation and, accordingly, proposed the application of catechin to stabilize diseased connective tissue. The protein-stabilizing properties of phenolic tannins (also referred to bioflavenoids or catechins) have been well documented in the literature. See, e.g., Cetta, “Influence of Flavenoid-Copper Complexes on Cross-Linking in Elastin,” Ital. J. Biochem., 1977; Heijmen, “Cross-linking of Dermal Sheep Collagen with Tannic Acid,” Biomaterials, v. 18, 1997; Koide, “Effect of Various Collagen Cross-Linking Techniques on Mechanical Properties of Collagen Film,” Dental Materials Journal, v. 16(1), 1997; Lier, “Review of the Scientific Research on Pycnogenols,” www.integratedhealth.com/infoabstract/pycdes.html, 2003; Han, “Pranthocyanidin: A Natural Cross-Linking Reagent for Stabilizing Matrices,” J. Biomed. Mater. Res., 2003.

Additionally, Schreck (U.S. Patent Application Publication No. 2004/0230156) proposed in-situ cross-linking of vascular tissue to protect against diseases involving enzymatic degradation of the vessel wall including vulnerable plaque and aortic aneurysms. Schreck disclosed catheter-based delivery systems to deliver the cross-linking agent into the vessel wall. Vyavahare (U.S. Pat. No. 7,252,834) proposed direct application of phenolic tannins to aneurismal aortic tissue to cross-link the elastin in the extracellular matrix.

Localized pharmacological interventions to stabilize aortic tissue against further degradation by enzymes require delivery of the agent into the vessel wall. Endovascular approaches are sometimes preferred due to their minimally invasive nature. However, the endovascular approach has two major challenges. To avoid immediate wash-out of the agent, the vessel wall may need to be isolated from the blood stream during drug delivery. The literature indicates that an application of a suitable cross-linking agent for at least 10-15 minutes may be required to achieve noticeable cross-linking of elastin and collagen. Even longer application may be required to down-regulate processes associated with the degradation of the tissue such as inflammation and expression and activation of enzymes including MMPs. Delivery of the agent with a balloon catheter, as proposed in some embodiments of U.S. Patent Application Publication No. 2004/0230156 and U.S. Pat. No. 7,252,834, occlude the blood vessel during the application of the agent. This approach may be reasonable in applications in the abdominal aorta but not suitable for application in the thoracic aortic due to the high flow rates and blood pressure acting on the balloon. A second challenge is the presence of mural thrombus on the luminal surface of the diseased vessel. Mural thrombus typically lines the sac of aortic aneurysms and the false lumen of dissections. The thrombus typically grows as the disease progresses and can reach a thickness of several centimeters. The delivery systems and methods referenced above do not take into consideration the barrier created by thrombus.

### SUMMARY OF SOME EMBODIMENTS

Some embodiments of the present disclosure are directed to a method for stabilizing an extracellular matrix in a wall of a blood vessel comprising advancing a delivery system to a treatment site positioned near a mural thrombus that covers at least a portion of the wall of the blood vessel. A delivery portion of the delivery device is advanced into the mural thrombus. A therapeutic agent is delivered through the delivery portion into the mural thrombus. The agent can transport from the mural thrombus into the extracellular matrix of the vessel wall by diffusion to facilitate reduction of enzymatic degradation of protein in the extracellular matrix by the action of the agent.

Additionally, some embodiments of the present disclosure are directed to a method for stabilizing an extracellular matrix layer in the vascular system of a body, comprising positioning a portion of a vascular catheter adjacent to or within a mural thrombus positioned adjacent to the extracellular matrix layer of a target region of the vascular system. A therapeutic agent is delivered in solution to the mural thrombus using the vascular catheter. The therapeutic agent can be transported to the extracellular matrix layer through the mural thrombus to promote the cross-linking protein in the extracellular matrix layer, thereby stabilizing the extracellular matrix.

Some embodiments of the present disclosure are directed to a catheter system for the delivery of a therapeutic agent into a wall of a blood vessel, comprising a delivery catheter. The delivery catheter houses a therapeutic agent configured to promote the cross-linking of protein. The catheter system also comprises a delivery portion of the delivery catheter. The delivery portion is configured to deliver the therapeutic agent from the delivery catheter into a mural thrombus.

### BRIEF DESCRIPTION OF THE DRAWINGS

These and other features, aspects and advantages of the present disclosure will now be described in connection with non-exclusive embodiments, in reference to the accompanying drawings. The illustrated embodiments, however, are merely examples and are not intended to limit the invention. The following are brief descriptions of the drawings, which may not be drawn to scale.

FIG. 1 illustrates the molecular structure of various catechins.

FIG. 2 illustrates mural thrombus in an aneurysm.

FIG. 3 illustrates an embodiment of a delivery system for the delivery of an agent into the mural thrombus.

FIG. 4 illustrates the end or tip portion of the embodiment of the delivery system illustrated in FIG. 3.

FIG. 5 is a partial sectional side elevational view of one arrangement of a drug delivery and temporary stent catheter.

FIG. 6 is a cross sectional view taken along the lines 2-2 of FIG. 5.

FIG. 7 is a partial sectional side elevational view of another arrangement of a catheter, having a coaxially configured catheter body.

FIG. 8 is a cross-sectional view taken along the lines 4-4 in FIG. 7.

FIG. 9 is a partial sectional side elevational view of an over-the-wire arrangement of a catheter.

FIG. 10 is a partial sectional side elevational view of a non-stent arrangement of a catheter.

FIG. 11 is a cross-sectional view taken along the lines 7-7 in FIG. 10.

FIG. 12 is a cross-sectional view taken along the lines 8-8 in FIG. 10.

FIG. 13 is a cross-sectional view taken along the lines 9-9 in FIG. 10.

FIG. 14 is a side elevational view of a non-stent arrangement in communication with a fluid delivery and guidewire entry apparatus.

FIG. 15 is a perspective view of the non-stent embodiment the catheter.

FIG. 16 is a side view of an embodiment of an angioplasty balloon catheter with a semi-elastic balloon that is loaded with a therapeutic agent.

FIG. 17A is a side view of an embodiment of an angioplasty balloon catheter with a PTA balloon covered by a tubular sleeve that is loaded with a therapeutic agent in a collapsed position.

FIG. 17B is the embodiment of the balloon catheter of FIG. 17A, partially inflated.

FIG. 17C is the embodiment of the balloon catheter of FIG. 17A, fully inflated.

FIG. 18A is a side view of an embodiment of an angioplasty balloon catheter with an inner PTA balloon and an outer balloon loaded with a therapeutic agent and the balloon in a collapsed position.

FIG. 18B is a side view of the embodiment of the angioplasty balloon catheter of FIG. 18A with the balloon fully inflated.

FIG. 19A is a side view of an embodiment of an angioplasty balloon catheter with an inner PTA balloon and an outer balloon loaded with a therapeutic agent, the inner balloon being axially positioned within the outer balloon to inflate segments of the outer balloon. FIG. 19A shows the inner balloon inflating a distal section of the outer balloon.

FIG. 19B shows the embodiment of the angioplasty balloon catheter of FIG. 19A, showing the inner balloon inflating the proximal section of the outer balloon.

FIG. 20A shows an SEM image at 5.0 k magnification of a latex surface prepared with 1% Polyethylene glycol (PEG) having molecular weight of between approximately 380 and approximately 420.

FIG. 20B shows an SEM image at 5.0 k magnification of the surface of the latex shown in FIG. 20A, stretched to about 400% of its original dimensions.

### DETAILED DESCRIPTION OF SOME EXEMPLIFYING EMBODIMENTS

Disclosed herein are various embodiments of a novel apparatus for and a novel method of delivering a pharmacological agent into the wall of diseased blood vessels. Specifically, in some embodiments, the apparatuses and the methods are adapted for the treatment of an aneurysm and/or dissections in which a significant mural thrombus is present.

Several studies suggest that the mural thrombus is an indicator of active tissue degeneration. See, e.g., Vorp, “Association of Intraluminal Thrombus In Abdominal Aortic Aneurysm With Local Hypoxia And Wall Weakening,” J. Vasc. Surg., 2001; Wolf, “Computed Tomography Scanning Findings Associated With Rapid Expansion Of Abdominal Aneurysms,” J. Vasc. Surg., 1994; Satta, “Intraluminal Thrombus Predicts Rupture Of An Abdominal Aortic Aneurysm,” J. Vasc. Surg., 1996; Bonser, “Clinical And Patho-Anatomical Factors Affecting Expansion Of Thoracic Aortic Aneurysms,” Heart, 2000; Tsai “Partial Thrombosis Of The False Lumen In Patients With Acute Type B Aortic Dissections,” N. Engl. J. Med. 2007. Mural thrombus has been identified as a risk factor for aortic rupture. The mural thrombus is a site of platelet aggregation, inflammatory processes, expression of MMP, and enzymatic degradation, which all contribute to the degeneration of the extra-cellular matrix in the aortic wall. Aneurysmal aortic wall segments that are covered by thrombus are typically thinner and more likely to rupture than uncovered segments. The fluid layer at the interface between the mural thrombus and the vessel wall can have a high concentration of enzymes involved in the degeneration of the tissue. Furthermore, the thrombus can reduce the transport of oxygen to the endothelial cells causing hypoxia and cell death. Thus, the mural thrombus plays an important role in the degenerative aortic disease, but has not been considered a target for therapeutic methods or apparatuses.

In some embodiments of the present disclosure, the thrombus is utilized as a delivery matrix for a therapeutic agent targeted to stabilize the vessel wall. In addition, the therapeutic agent may also inhibit the degenerative processes that take place in the mural thrombus. Preferably, in some embodiments, the agent can be injected or otherwise delivered into the abluminal wall layers of the thrombus. Once the agent has been delivered, the thrombus can retain the agent and prevent immediate wash-out of the agent into the blood stream. At the same time, the high permeability of the thrombus can facilitate diffusion of the agent throughout the thrombus and into the vessel wall. The fluid interface between the thrombus and the vessel wall can provide a channel for rapid transport of the agent along the surface of the vessel wall and uniform distribution of the agent relative to the vessel wall adjacent to the thrombus.

Because the mural thrombus is typically soft in some embodiments, the mural thrombus can be penetrated with a needle or small-profile injection catheter. Other delivery apparatuses and methods can be used, as will be discussed. The injection of an agent can be guided by fluoroscopy, MRI, echocardiography, or any other suitable imaging modalities. Contrast medium can be added to the solution containing the agent to visualize the injection of the agent. In some embodiments, the agent can be delivered endovascularly with a catheter-based delivery system. Alternatively, the agent can be delivered percutaneously through the skin. For example, a trans-lumbar needle puncture can be utilized to access the thrombus in an abdominal aortic aneurysm from the back of the patient. Since the thrombus may cover a large portion of the aneurysm, multiple injections may be preferred to treat larger regions of the diseased vessel.

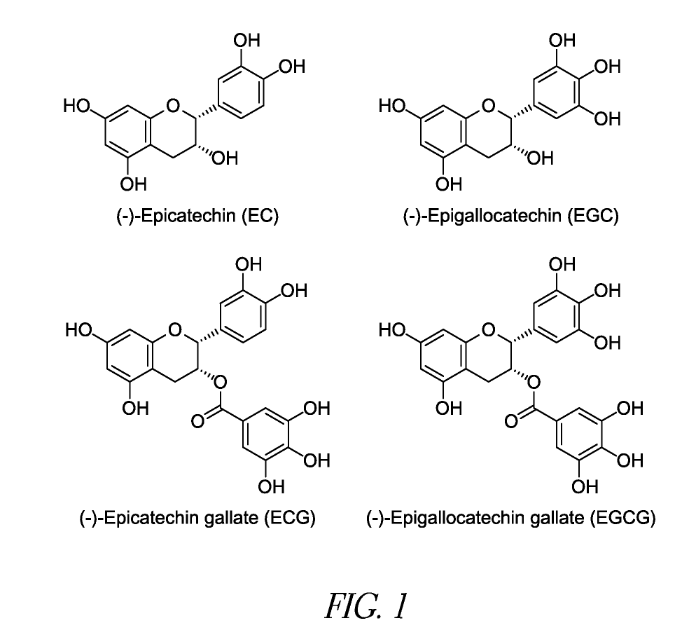

Suitable agents may include anti-inflammatory agents, MMP inhibitors, and collagen and elastin cross-linking agents. In some embodiments, catechins can be used as the therapeutic agent. The chemical structure of catechin is shown in FIG. 1. Catechins have anti-platelet and anti-inflammatory properties. Catechins can inhibit matrix-metalloproteinaneses (MMPs) and can up-regulate collagen synthesis. Catechin delivered into the thrombus can also disrupt the inflammatory and enzymatic activities in the thrombus, which can contribute to the degradation of the extra-cellular matrix in the vessel wall. In sufficiently high concentrations, catechins can cross-link elastin and/or collagen and can protect the proteins against enzymatic degradation. Catechins are readily soluable in aqueous solution, have a low level of toxicity, and demonstrate a high affinity to collagen, which is one of the target proteins in the extracellular matrix of the vessel wall. Catechin can also promote the synthesis of collagen in the aortic wall to replace degraded collagen. Suitable catechins for this application can include Epicatechin (EC), Epigallocatechin-3-gallate (EGCG), Epigallocatechin (EGC), and Epicatechin-gallate (ECG). EGCG is the most potent cross-linking agent of the catechin family. However, a combination of EGCG, ECG, and EGC may have synergistic, anti-inflammatory and anti-platelet effects. In some embodiments, a composition of catechins comprising at least 40% EGCG can be used. In some embodiments, the agent can be a proanthocyanidin, quercetin, tannic acid, or any combination thereof.

In the following sections, more detailed embodiments are described for a catheter for delivering catechins. However, the methods, apparatuses, and devices disclosed herein are not limited to use for delivering catechin but can be used or adapted for any suitable agent that is desired to be delivered into the mural thrombus of diseased arteries.

Catechin can be delivered in solution form into the mural thrombus. This delivery method pathway can be used, without limitation, when a short-acting application of a high-concentration of catechin is desired. Experiments conducted by the author indicate that short-term (approximately 10-30 minute) application of catechin to vascular tissue can stabilize the collagen contained within the vascular tissue. In some embodiments, the concentration of the agent can be in the range from approximately 0.01% to approximately 20%, or between approximately 2.0% and approximately 10.0%, or to or from any values within these ranges.

In some embodiments, the bioflavonoid EGCG or other suitable agents can be delivered in a pH-buffered solution (e.g. phosphate buffer) of approximately pH 7.4 to minimize damage to living tissue. In some embodiments, the pH of the solution can be altered in order to optimize the reaction kinetics. For example, hydrogen bonds form about 50% faster when the pH is reduced to about 4.0. The reaction kinetics may be affected by the pH of the treatment solution compared to the isoelectric point of the protein to be cross-linked. In some arrangements, the pH of the solution can be matched to the isoelectric point of collagen (approximately pH 5.6-5.8) or elastin (approximately pH 4.0) in the vessel. To improve penetration of the agent into the tissue and minimize swelling of the tissue during fixation, a keotropic agents such as Ca(OH)2 or Dimethyl Salfoxite (DMSO) can be added to the solution. To visualize the delivery of the agent, a radiopaque contrast agent can be added to the solution. Diffusion and soluability of catechin can be further facilitated by the addition of an organic solvent to the solution. Examples of organic solvents are acetone, alcohol, ethyl acetate, methanol, and methyl acetate. In some embodiments, the concentration of the solvent can be in the range of about 1% to about 90%, or in the range of about 10% to about 50%, or to or from any values within these ranges.

In an alternative embodiment, catechin or other suitable agents may be delivered within a delivery matrix to facilitate slow release of the agent. Without limitation, this approach can be used when the inflammatory and enzymatic processes in the thrombus and the aortic wall are targeted, which may require a lower therapeutic dose over a longer period of time. Suitable microcarrier matrices can include biodegradable polymers or hydrogels, which are well described in the literature.

It will be obvious to the reader skilled in the art that there are various methods to deliver a therapeutic agent. One novel aspect of this disclosure is the utilization of the mural thrombus as a matrix to deposit or infuse the agent that targets the vessel wall. The advantage of this approach is that the soft thrombus can be readily penetrated and the agent can be delivered in a very short period of time via injection. The vessel wall will not be impacted or damaged by this delivery method. Once delivered, the agent can be generally protected from immediate wash-out by the blood and can migrate to the vessel wall.

Additionally, in some embodiments, long-term therapeutic action of the agent is possible. The agent can be delivered in high concentrations that cause local cell death since cells contained in the thrombus may not be critical for the viability of the vessel wall. To the contrary, cells contained in the thrombus are typically associated with degenerative and inflammatory processes. Thus, the agent can be injected in high concentrations that are typically not suited for direct injection into the vascular tissue. Therapeutic formulations of catechin typically have a concentration of catechin in the range of about 0.01% to 0.1%. In some embodiments, the concentration of the catechin can be from about 1% to about 20%. For example, in some embodiments, injection of one or more therapeutic agents (such as, but not limited to, catechin) into the thrombus in sufficiently high concentrations can beneficially reduce the concentration of platelets, inflammatory cells, or other detrimental compounds in the thrombus. Additionally, injection of one or more therapeutic agents (such as, but not limited to, catechin) into the thrombus in sufficiently high concentrations can beneficially mitigate the build up of thrombus adjacent to the vessel wall.

The mechanical properties of soft thrombus are very different from that of the aortic wall. Thrombus is a fibrin structure with blood cells, blood proteins, and cellular debris (Van Dam, “Non-linear viscoelastic Behavior Of Abdominal Aortic Aneurysm Thrombus,” Biomechanics and Modeling in Mechanobiology, 2008). New thrombus is typically formed on the luminal side with well organized fibrin structures. The abluminal (wall) layer of the thrombus is typically older with less organized structures. This may be due to the enzymatic breakdown of protein in this region. The thrombus can be very soft and elastic and can easily be penetrated with a blunt object. Conversely, the aortic wall includes an extracellular matrix of collagen and elastin protein capable of withstanding the high tensile forces imposed by the blood pressure. The differences in the material properties of the thrombus and the aortic wall can be utilized to design a delivery system that penetrates into the abluminal layer of the thrombus but does not penetrate or damage the underlying aortic wall.

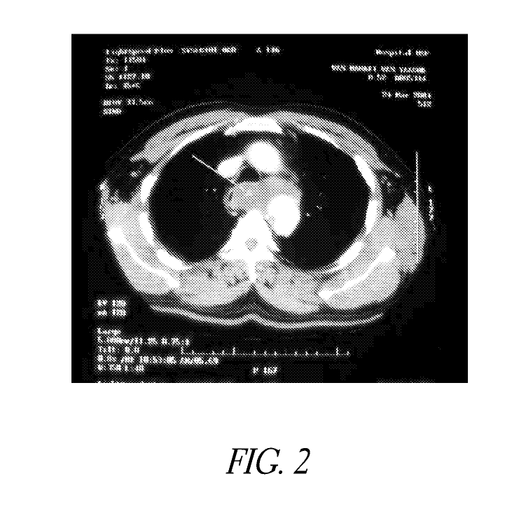

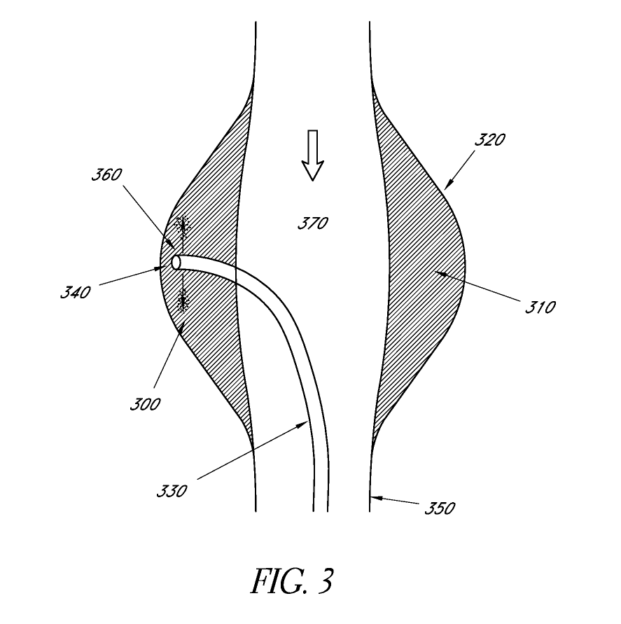

FIG. 2 shows a CT image of an abdominal aortic aneurysm. The mural thrombus is indicated by the arrow. The mural thrombus fills a significant cross-section of the aneurysm. FIG. 3 illustrates an embodiment of a delivery system for delivering the agent 300 or drug into the mural thrombus 310. In the illustration, the aorta is shown with a large aneurysm 320 and the mural thrombus 310 partially or fully fills the sac of the aneurysm 320. The delivery system comprises an injection or delivery catheter 330 that can be advanced through a puncture site in the femoral artery or any other suitable vessel and advanced so that a delivery portion 340 of the catheter 330 can be inserted into the abluminal portion of the thrombus 310. The delivery portion 340 can be atraumatic to protect the vessel wall 350 from injury. In the illustrated embodiment, the delivery portion 340 can be positioned near the end or tip of the delivery catheter 330. However, in other embodiments, the delivery portion 340 can be distanced from the end or tip of the delivery catheter 330 and or can be positioned at or near the tip of the catheter in addition to being distanced from the end or tip of the delivery catheter 330.

In some embodiments, the agent 300 or drug can be injected or otherwise delivered into the thrombus 310 by ejecting the agent 300 from side ports 360 in the catheter 330 parallel, transverse, or at any orientation relative to the vessel wall 350 to distribute the agent 300 along the vessel wall 350. The thrombus 310 can help diffuse the agent 300 over a larger area of the vessel wall 350 or extracellular matrix and thereby stabilize the aortic wall tissue. Therefore, injecting the therapeutic agent 300 in a high concentration into the thrombus 310 can beneficially diffuse or distribute the therapeutic agent 300 over a wider area of the surface of the vessel wall 350 as compared to directly injecting the therapeutic agent 300 into the vessel wall 350 or extracellular matrix. This can minimize the number of injections that would otherwise be required to treat an area of the extracellular matrix, thus reducing the risk of rupturing or otherwise injuring the vessel wall 350 which can occur from multiple injections.

Furthermore, by using the delivery catheter 330 apparatus or method described below or other embodiments disclosed herein, the therapeutic agent 300 can be administered to the thrombus 310 without the use of a syringe, thereby reducing the risk of rupturing or otherwise injuring the vessel wall 350. Finally, because the thrombus 310 can act as a reservoir for the therapeutic agent 300 whereby the agent 300 is inhibited from washing out of the thrombus 310 into the blood stream 370, the agent 300 can be delivered in a localized nature so that the exposure of other body tissue to the agent 300 can be controlled.

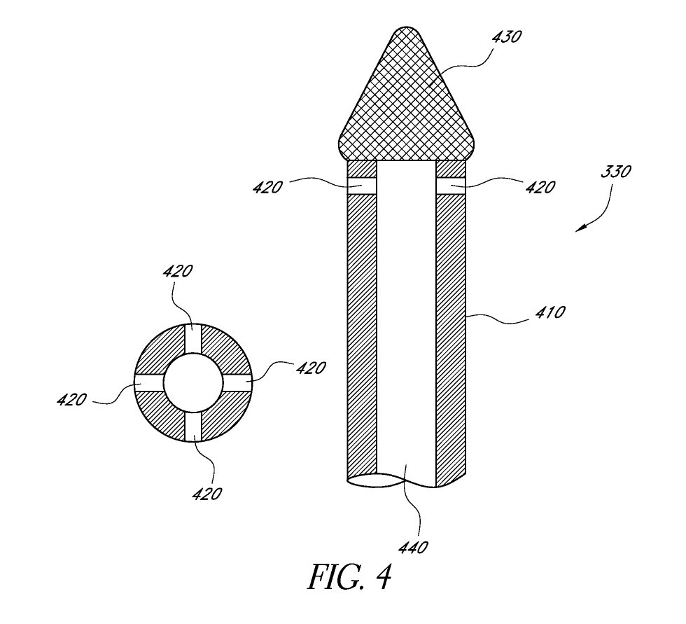

FIG. 4 illustrates one embodiment of an end portion 410 of the embodiment of the delivery catheter 330 of FIG. 3. In this embodiment, the end portion 410 includes an atraumatic tip 430. The agent can be transported from a reservoir (not shown) at the proximal end of the catheter 330 via a delivery lumen 440 to the tip 430 of the catheter 330. The catheter 330 can have a blunt and soft tip 430 to prevent damage to the aortic wall. The tip 430 can be radiopaque or a radiopaque marker may be placed in the tip for visualization. The ejection ports 420 can be placed on the side of the catheter 330 to eject the agent parallel to the aortic wall. The delivery catheter 330 can have a small profile for percutaneous insertion into the artery. In some embodiments, the crossing profile of the delivery catheter 330 can be less than 12 French. In some embodiments, the crossing profile of the delivery catheter can be less than 8 French or between approximately 8 French or less and approximately 12 French. In some embodiments, the delivery catheter can have a lumen to house a guidewire for “over-the-wire” delivery. In some embodiments, the tip of the catheter can be articulated. It will be obvious to the reader familiar with catheter-based delivery systems that there are many potential alternative embodiments for a steerable catheter, which are contemplated herein.

The agent can be injected into the catheter with a syringe. Alternatively, a high-pressure needleless injection system may be used to deliver the agent into the thrombus or into the tissue. The advantage of such a high-pressure injector system is that the agent can be injected over a larger area in the thrombus, providing a more even and faster delivery. In some embodiments, the agent can be injected into the thrombus with a syringe.

In order to investigate the possibility of administering catechins into the thrombus and achieving collagen stabilization, a thrombus model was developed. The model consisted of fresh bovine blood which had been preserved with EDTA as an anti-coagulating agent and fresh bovine pericardium. Bovine pericardium has a high collagen content. Inert plastic trays were used to contain the blood and pericardium, which was placed on the tray bottom. The blood was coagulated by exceeding the chelation capability of the EDTA with Ca++ ions. The thrombus created was uniform and approximately 1 cm thick. The thrombus was allowed to mature several hours before testing was commenced. There were four experiments conducted with this thrombus and pericardium model. Polyphenon (Polyphonen E International) was used as a cross-linking agent. Polyphenon E (PPE) consists of EGCG, ECG, and EGC. The concentration of EGCG was at least 40%. The scope of the experiments was to determine if PPE could be injected into the thrombus present within an aortic aneurysm and from this injection provide stabilization of the collagen in the region of the thrombus. Tissue stabilization was determined from the temperature at which tissue samples shrunk by 10% in length (shrinkage temperature Ts). Increases in shrinkage temperature of treated collagen tissue versus untreated collagen tissue is an indication of tissue stabilization.

Experiment 1—Model Development and Feasibility Testing: The initial experiment consisted of depositing a thrombus layer over a pericardium sample, injecting an aqueous solution of PPE into the thrombus at multiple sites and allowing the PPE time to react with the pericardium. The samples were examined for cross-linking of the collagen and changes to the thrombus associated with the PPE injection. All pericardium samples exhibited increased shrinkage temperature values indicative of increased collagen stabilization.

| | Shrinkage Temperature (° C.) | | | | | | | | | |
|---|---|---|---|---|---|---|---|---|---|---|
| | 1 | 2 | 3 | 4 | 5 | 6 | Ave | STD | MAX | Min |
| Run 1 | 74.6 | 76.5 | 75.7 | 73.2 | 70.9 | | 74.2 | 2.21 | 76.5 | 70.9 |
| Run 2 | 73.4 | 72.8 | 70.7 | 70.8 | 70.1 | 70.1 | 71.3 | 1.42 | 73.4 | 70.1 |
| Run 3 | 72.0 | 71.5 | 72.8 | 72.0 | 74.9 | 75.6 | 73.1 | 1.71 | 75.6 | 71.5 |
| Run 4 | 75.1 | 77.2 | 76.8 | 76.4 | 73.8 | 73.8 | 75.5 | 1.51 | 77.2 | 73.8 |
| Native | 69.3 | 67.7 | 68.6 | 69.1 | 68.5 | 68.9 | 68.7 | 0.57 | 69.3 | 67.7 |

Experiment 2—Solvent Delivery System: The second experiment used the thrombus pericardium system developed in the initial experiment. The aqueous PPE solution was replaced with an ethanol:water PPE solution. Controls for interaction of the pericardium with the ethanol water solution and thrombus were performed as part of this experiment. The 40:60 ethanol:water solution allowed for a greater PPE concentration (20% compared to 15% in water) but the increase in shrinkage temperatures observed were lower for the ethanol:water PPE solution than those found for PPE in water. The controls indicated no interactions between either the ethanol:water solution or the thrombus and the pericardium with respect to changes in the collagen shrinkage temperature.

| | | Shrinkage Temperature (° C.) | | | | | | | | | |
|---|---|---|---|---|---|---|---|---|---|---|---|
| | Treatment | 1 | 2 | 3 | 4 | 5 | 6 | Ave | STD | MAX | Min |
| Run 1 | EtOH | 68.2 | 67.6 | 66.7 | 67.3 | 67.5 | 67.6 | 67.5 | 0.49 | 68.2 | 66.7 |
| Run 2 | PPE | 70.0 | 71.0 | 71.4 | 70.0 | 70.9 | 70.4 | 70.6 | 0.57 | 71.4 | 70.0 |
| Run 3 | PPE | 72.4 | 72.4 | 73.0 | 72.7 | 73.9 | 73.0 | 72.9 | 0.56 | 73.9 | 72.4 |
| Run 4 | Blood | 68.2 | 68.1 | 68.2 | 67.6 | 67.8 | 68.1 | 68.0 | 0.24 | 68.2 | 67.6 |
| Run 5 | EtOH | 67.7 | 67.8 | 67.8 | 67.4 | 67.4 | 67.3 | 67.6 | 0.23 | 67.8 | 67.3 |
| Run 6 | Blood | 68.9 | 69.1 | 69.4 | 69.1 | 68.9 | 68.9 | 69.1 | 0.20 | 69.4 | 68.9 |

| | | | | | | | | | | |
|---|---|---|---|---|---|---|---|---|---|---|
| Native Control | 68.2 | 67.3 | 68.1 | 67.3 | 69.4 | 68.6 | 68.2 | 0.80 | 69.4 | 67.3 |

Experiment 3—Minimal Exposure Time: The third experiment measured the increases in shrinkage temperature after minimal exposure times. The pericardium/thrombus samples were prepared as in the initial two experiments. An aqueous, 14% PPE injection solution was used. Exposure was terminated after fifteen, thirty and sixty minutes and the collagen shrinkage temperature evaluated. There was little change after fifteen minutes, the largest average change after 30 minutes and the largest individual increase after 60 minutes. The large variation between samples within the same pericardium sample indicates an uneven distribution of PPE to the pericardium.

| | | Shrinkage Temperature (° C.) | | | | | | | | | |
|---|---|---|---|---|---|---|---|---|---|---|---|
| | Time | 1 | 2 | 3 | 4 | 5 | 6 | Ave | STD | MAX | Min |
| Run 1 | 1 hour | 68.7 | 69.6 | 71.6 | 74.0 | 75.1 | 79.3 | 73.1 | 3.93 | 79.3 | 68.7 |
| Run 2 | 30 min | 72.1 | 74.0 | 73.0 | 78.7 | 77.3 | 73.1 | 74.7 | 2.66 | 78.7 | 72.1 |
| Run 3 | 15 min | 69.3 | 70.1 | 70.3 | 70.1 | 70.4 | 69.1 | 69.9 | 0.55 | 70.4 | 69.1 |

| | | | | | | | | | | |
|---|---|---|---|---|---|---|---|---|---|---|
| Native Control | 68.1 | 68.0 | 69.0 | 68.9 | 68.8 | 69.0 | 68.6 | 0.46 | 69.0 | 68.0 |

Experiment 4—Evaluation of Delivery and Imaging Systems: The fourth experiment utilized samples similar to those used in the first three experiments. A delivery system was created consisting of a narrow gauge tube (5 Fr hollow catheter) with multiple holes with the end of the lumen plugged. Sufficient intact tubing was included to allow insertion of the irrigation portion of the catheter through the thrombus and into the interface between the thrombus and pericardium. A contrast media was used to image the injection of PPE in real time. A 19.4% PPE solution was used and diluted 50% with the contrast media. An open ended non-irrigation catheter and injections without contrast agent were also tested as controls. The contrast media had a positive impact on the collagen shrinkage temperature results. Samples treated with the PPE—contrast media solution had higher and more uniform increases in collagen shrinkage temperature than did the same catheters without the contrast media.

| PPE | Shrinkage Temperature (° C.) | | | | | | | | | |
|---|---|---|---|---|---|---|---|---|---|---|
| Treatment | 1 | 2 | 3 | 4 | 5 | 6 | Ave | STD | MAX | Min |
| Open End | 71.7 | 76.9 | 78.3 | 78.0 | 78.3 | 74.0 | 76.2 | 2.74 | 78.3 | 71.7 |
| contrast | | | | | | | | | | |
| 1st Irrigator | 79.2 | 78.4 | 79.2 | 79.4 | 77.0 | 75.6 | 78.1 | 1.53 | 79.4 | 75.6 |
| Contrast | | | | | | | | | | |
| 2nd Irrigator | 79.9 | 79.7 | 79.2 | 79.0 | 79.4 | 80.0 | 79.5 | 0.40 | 80.0 | 79.0 |
| Contrast | | | | | | | | | | |
| 1st Irrigator | 75.6 | 79.8 | 76.0 | 70.2 | 69.0 | 69.6 | 73.4 | 4.40 | 79.8 | 69.0 |
| No Contrast | | | | | | | | | | |
| 2nd Irrigator | 74.7 | 71.2 | 69.9 | 69.7 | 69.5 | 69.9 | 70.8 | 1.99 | 74.7 | 69.5 |
| No Contrast | | | | | | | | | | |
| No Contrast, No | 67.9 | 68.0 | 68.3 | 68.5 | 68.5 | | 68.2 | 0.28 | 68.5 | 67.9 |
| PPE | | | | | | | | | | |

Furthermore, other known apparatuses and methods may be suitable for injecting, diffusing, or otherwise delivering the agent to the thrombus and/or tissue, and are contemplated as being a part of the present disclosure. For example, without limitation, the apparatuses, methods, and/or therapeutic agents disclosed in the patent applications incorporated by reference above as if fully set forth herein disclose apparatuses and/or methods that are suitable for injecting, diffusing, or otherwise delivering the agent to the thrombus, as well as various therapeutic agents that may be suitable for delivery by any of the methods or apparatuses disclosed herein, including the disclosure of the patent applications incorporated by reference herein. The applications that are incorporated by reference in their entireties herein include U.S. Provisional Patent Application 60/987,261, filed Nov. 12, 2007, U.S. Provisional Patent Application 61/012,356, filed Dec. 7, 2007, U.S. Provisional Patent Application 61/127,654, filed May 14, 2008, U.S. Provisional Patent Application 61/012,579, filed Dec. 10, 2007, U.S. Provisional Patent Application No. 60/533,443, filed on Dec. 31, 2003, and U.S. patent application Ser. No. 12/269,677, filed Nov. 12, 2008.

### Therapeutic Agent Delivery Methods

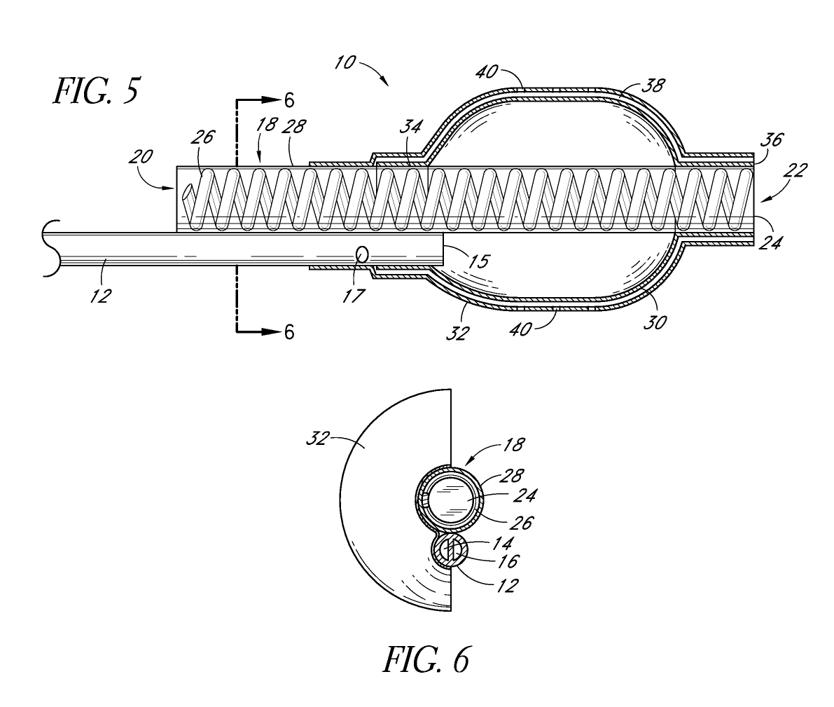

FIGS. 5-15 describe various embodiments of a drug delivery catheter and dilation catheter, which can be used to deliver a therapeutic agent to the thrombus or vessel wall. The embodiments of the drug deliver catheter are described in additional detail in U.S. Pat. No. 5,295,962 to Crocker et al., the entire contents of which are hereby incorporated by reference herein.

Referring to FIG. 5, there is illustrated a combination drug delivery and temporary stent catheter. Although the illustrated embodiment incorporates both the drug delivery and temporary stent features, catheters incorporating only the drug delivery feature or a drug delivery feature in combination with another therapeutic procedure or device can also be readily produced in accordance with the disclosure herein, as will be appreciated by one of skill in the art. In addition, the catheter can readily be used for angioplasty dilation as well.

The catheter 10 of the illustrated embodiment can comprises an elongate tubular body 12 for extending between a proximal control end (not illustrated) and a distal functional end. Tubular body 12 can be produced in accordance with any of a variety of known techniques for manufacturing balloon tipped catheter bodies, such as by extrusion of appropriate biocompatible plastic materials. Alternatively, at least a portion or all of the length of tubular body 12 can comprise a spring coil, solid-walled hypodermic needle tubing, or braided reinforced wall, as is well understood in the catheter and guidewire arts.

Tubular body 12 can have a generally circular cross-sectional configuration having an external diameter within the range of from about 0.030 inches to about 0.065 inches. Alternatively, a generally triangular cross-sectional configuration can also be used, with the maximum base to apex distance also within the range of from about 0.030 inches to about 0.065 inches. Other non-circular configurations such as rectangular or ovular can also be used. In peripheral vascular applications, the body 12 can have an outside diameter within the range of from about 0.039 inches to about 0.065 inches. In coronary vascular applications, the body 12 will typically have an outside diameter within the range of from about 0.030 inches to about 0.045 inches.

Diameters outside of the aforemention ranges can also be used, provided that the functional consequences of the diameter are acceptable for a specified intended purpose of the catheter. For example, the lower limit of the diameter for tubular body 12 in a given application can be a function of the number of fluid or other functional lumen contained in the catheter, together with the acceptable flow rate of dilation fluid or drugs to be delivered through the catheter.

In addition, tubular body 12 can be configured to have sufficient structural integrity (e.g., “pushability”) to permit the catheter to be advanced to distal arterial locations without buckling or undesirable bending of the tubular body 12. The ability of the body 12 to transmit torque may also be desirable, such as in embodiments having a drug delivery capability on less than the entire circumference of the delivery balloon. Larger diameters can have sufficient internal flow properties and structural integrity, but reduce perfusion in the artery in which the catheter is placed. In addition, increased diameter catheter bodies tend to exhibit reduced flexibility, which can be disadvantageous in applications requiring placement of the distal end of the catheter in a remote vascular location.

With reference to FIG. 6, the tubular body 12, in accordance with the illustrated embodiment, comprises at least a first lumen 14 and a second lumen 16 extending axially therethrough. Inflation lumen 14 can be in fluid communication with the interior of inflation balloon 30 by way of port 15. Drug delivery lumen 16 can be in fluid communication with a drug delivery balloon 32 by way of port 17. In this manner, inflation fluid or fluid medication can be selectively introduced into the inflation balloon 30 and drug delivery balloon 32, as will be described in greater detail below.

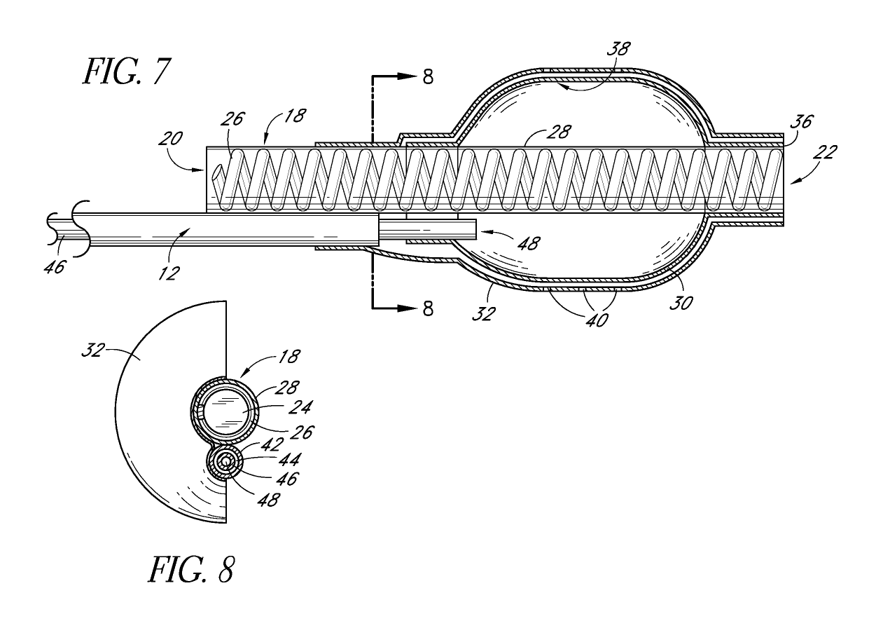

Additional lumen can readily be formed in tubular body 12 by techniques known in the art. In one embodiment (not illustrated), a third lumen is provided having an opening at its proximal end and a closed distal end. This third lumen receives a wire to improve pushability of the catheter. A further embodiment, illustrated in FIG. 9 and discussed infra, is provided with a guidewire lumen for over-the-wire manipulation.

In a modified embodiment of the catheter body, two or more lumens are disposed in a concentric arrangement. See FIGS. 7 and 8. Tubular body 12 comprises an outer tubular wall 42 defining a first lumen 44 for communicating a fluid to the distal end of the catheter. An inner tubular wall 46 defines a second lumen 48. In the illustrated embodiment, inner lumen 48 can be in fluid communication with the inflation balloon 30, and outer lumen 44 can be in fluid communication with the drug delivery balloon 32. Concentric lumen catheter bodies can be manufactured in accordance with techniques known in the art.

A temporary stent 18 can be secured to the distal end of tubular body 12. As illustrated in FIG. 5, the longitudinal axis of temporary stent 18 can be laterally displaced from the longitudinal axis of tubular body 12. Stent 18 can comprise a first end 20, a second end 22 and a lumen 24 extending therebetween as shown in FIG. 6. Blood flow through lumen 24 can occur in either direction, depending upon the location of percutaneous insertion and the direction of transluminal travel of the catheter.

In general, the ratio of the interior cross-sectional area of lumen 24 to the maximum exterior cross-sectional area of the deflated balloon can be maximized in order to optimize perfusion across the inflation balloon 30 while inflation balloon 30 is inflated. Catheter arrangements having a perfusion deflated profile of 0.055 inches or greater can be produced having an interior lumen 24 with an interior diameter of at least about 0.030 inches, and, in another arrangement, about 0.039 inches or greater. This can fit readily within the lumen of a guide catheter, which can have an internal diameter of about 0.072 inches. Alternatively, the diameter of lumen 24 can be reduced to as low as about 0.012 inches and still function as a guidewire conduit.

In one embodiment, the interior diameter of lumen 24 can be about 0.039 inches (1 mm). This lumen can provide a flow at 80 mm Hg of greater than 60 ml/minute. The coil wall thickness of about 0.002 inches adds 0.004 inches to the diameter of stent 18. The outer sheath 28, described infra, can have a thickness of about 0.001 inches and can produce an assembled stent 18 having an outside diameter of about 0.045 inches.

The illustrated design can provide a significant passageway 24 cross sectional area compared to the overall cross sectional area of stent 18. This parameter can be advantageous because, in some embodiments, only the stent 18 and balloon will typically traverse the stenotic site. The distal end of catheter body 12 (i.e., port 15) can end proximally of the stenosis in the preferred application.

This parameter is conveniently expressed in terms of the percentage of the outside diameter of stent 18 that the thickness of a single wall of stent 18 represents. In other words, in a preferred embodiment, a 0.003 inch wall thickness is about 6.7% of the 0.045 inch outside diameter. In one arrangement, this percentage can be less than about 14%, and, in another arrangement, less than about 8%, and in another arrangement less than about 5% to optimize perfusion through the inflated balloon. Lower percentages may be achievable through the use of new materials or techniques not yet developed.

In some embodiments, lower percentages can be obtained by sacrificing pushability or by development or use of new high strength materials. For example, if sufficiently structurally sound for a given application, use of a 0.002 inch stent wall in a 0.045 inch diameter catheter will produce a 4.4% value. In addition, the percentage can be reduced by increasing the outside diameter of the stent to the maximum permitted for a given application.

Temporary stent 18 can comprise a support structure for resisting radial compression of passageway 24 by the inflated balloon 30. Suitable support structures include braided or woven polymeric or metal reinforcement filaments or a spring coil 26. Spring coil 26 can comprise a material having suitable biocompatibility and physical properties, such as a stainless steel or platinum wire. Alternatively, polymeric materials such as nylon or Kevlar (DuPont) can also be used. In one arrangement, rectangular ribbon can be used, having cross-sectional dimensions on the order of about 0.001 inches by about 0.003 inches for small vessels, and on the order of about 0.005 inches by about 0.010 inches for use in larger vessels.

The wire or ribbon can be wound to produce a coil having an interior diameter within the range of from about 0.030 inches (coronary) to about 0.100 inches (periphery) and an exterior diameter within the range of from about 0.032 inches (coronary) to about 0.110 inches (periphery).

Spring coil 26 can be either “tightly wound” so that adjacent loops of coils are normally in contact with each other, or “loosely wound,” as illustrated in FIG. 5, in which the adjacent loops of coil are normally separated from one another. The selection of a tightly wound or loosely wound coil for use in the present arrangement will be influenced by such factors as the desired weight of the finished catheter, the relative flexibility of the catheter in the region of temporary stent 18, and the amount of radially inwardly directed compressive force exerted by the inflation balloon 30, as will be apparent to one of skill in the art. Radiopacity may also be a factor.

A spring coil 26 can be provided with an outer sheath or coating 28. Sheath 28 can be produced by dipping, spraying, heat shrinking or extrusion techniques which are understood in the art, and can comprise a relatively flexible material having sufficient biocompatability to enable its use in contact with the vascular intima. Suitable materials for sheath 28 comprise linear low density polyethylene such as that produced by Dow, polyethylene terephthalate, nylons, polyester or other known or later developed medical grade polymers.

Inflation balloon 30 can comprise a proximal neck portion 34, a distal neck portion 36 and an intermediate dilation portion 38. Referring to FIGS. 5 and 7, it can be seen that the proximal neck of each balloon can be larger in diameter than the distal neck to accommodate the catheter body 12. Proximal neck portion 34 can be tightly secured to the temporary stent 18 and distal portion of tubular body 12, such as by the use of conventional adhesives, thermal bonding or heat shrinking techniques. The interstitial space formed by the diverging walls of tubular body 12 and temporary stent 18 (in a circular cross section embodiment) can be provided with a fluid-tight seal such as by filling with adhesive. In this manner, a fluid-tight seal between the proximal neck portion 34 and the elongate tubular body 12 and temporary stent 18 is provided.

The distal neck 36 of inflation balloon 30 can be provided with a fluid-tight seal with the distal portion of temporary stent 18. This seal can also be accomplished in any of a variety of manners known in the art, such as by the use of heat shrink materials, adhesives, or other thermal bonding or solvent bonding techniques. A distal neck 36 of inflation balloon 30 can in one arrangement be heat shrunk onto stent 18.

As will be appreciated by one of skill in the art, the sheath 28 can cooperate with the dilation portion 38 of the inflation balloon 30 to provide a sealed compartment for retaining a dilation fluid therein.

In some embodiments the inflation balloon can comprise a relatively non-elastic material such as linear low density polyethylene, polyethyleneterephthalate, nylon, polyester, or any of a variety of other medical grade polymers known for this use in the art. In some arrangements, the geometry, material and seals of balloon 30 can be configured to withstand an internal pressure of at least about 5 ATM and, other arrangements, about 10 ATM without any leakage or rupture.

The balloon 30 can be premolded to have an inflated diameter in a catheter intended for peripheral vascular applications within the range of from about 1.5 mm to about 8 mm. The balloon 30 in a catheter intended for coronary vascular applications can have an inflated diameter range of from about 1.5 mm to about 4 mm.

Although the illustrated embodiment has been described in terms of an “inflation” balloon 30, it is to be understood that the balloon 30 can also function as a dilation balloon, such as is well known in the art of percutaneous transluminal coronary angioplasty and other applications in which dilation of a stenotic region in a body lumen is desired. In an embodiment in which dilation properties are desired, conventional dilation balloon materials and design considerations can readily be incorporated, as will be understood by one of skill in the art. Alternatively, if the inflation balloon 30 is merely desired to provide sufficient radially expansive force to compress the drug delivery balloon 32 against the wall of the vessel, considerations appropriate for a lower pressure system may be utilized.

The drug delivery balloon 32 can be disposed radially outwardly from the inflation balloon 30. Drug delivery balloon 32 can comprise a generally non-elastic material such as is conventional for angioplasty dilation balloons, or may comprise an elastic material such as latex or urethane, or any other suitably biocompatible elastomer. Use of an elastic material for drug delivery balloon 32 can assist in reducing the relatively rough edges of the collapsed inflation balloon 30, and thereby reduce trauma to the vascular intima during insertion and withdrawal of the catheter.

Drug delivery balloon 32 can be provided with a plurality of delivery ports 40. Delivery ports 40 can be disposed radially symmetrically about the outer periphery of the delivery balloon 32, or can be limited to only portions of the exterior surface of the delivery balloon 32, depending upon the desired drug delivery pattern. For example, delivery ports 40 can be positioned only on one hemisphere of balloon 32. In another arrangement, delivery ports 40 can extend for less than the entire length of the balloon.

The delivery balloon 32 in a modified embodiment can comprise a material which is inherently permeable and/or porous, without the provision of discrete delivery ports 40. For example, woven or braided filaments or fabrics can be used. For relatively low delivery rate applications, fluid permeable membranes can also be used. In certain embodiments, the balloon 32 can be selectively permeable and/or porous, for example, made porous by the application of a release agent.

As can be seen with reference to FIG. 5, drugs or other agents or fluids introduced by way of lumen 16 can be expressed by way of port 17 into the interior space of drug delivery balloon 32. The inflated volume of inflation balloon 30 can cause the drug to be expelled by way of ports 40 outside of the drug delivery system.

In one arrangement, the relative inflated dimensions of the delivery balloon 32 and the inflation balloon 30 are such that a minimum amount of drug is retained between the two balloons. Thus, the inflated inflation balloon 30 can substantially completely fills the interior chamber of drug delivery balloon 32 to efficiently expel all or substantially all of the fluid introduced into drug delivery balloon 32 by way of drug delivery lumen 16. A residual volume of drugs contained in lumen 16 can be expelled outside of the balloon such as by following the drug with a small volume of normal saline or other “rinse” solution, as will be understood by one of skill in the art.

In a further arrangement, the inflation and drug delivery can be accomplished by the same balloon. In some embodiments, the permeability rate of the balloon material, or the diameter and number of delivery ports 40 can be sufficiently small that so the balloon is sufficiently firmly inflated without delivery at an excessive rate. Appropriate permeability rates for the balloon material can be determined through routine experimentation, in view of such factors as the viscosity of the drug, desired delivery rate and the desired radially expansive force to be exerted by the balloon.

Referring to FIG. 9, there is disclosed an over-the-wire embodiment of the delivery device. Over-the-wire catheter 50 can have a third lumen 52 extending through the housing 54. In one embodiment, housing 54 comprises a separate tube which is secured along the outside of catheter body 12 such as by adhesives or other plastic bonding techniques known in the art. In another arrangement, however, housing 54 can comprise an integrally formed three lumen catheter body as is well known in the art. Lumen 52 can be provided with a sufficient interior cross-sectional area to axially slidably receive a conventional guidewire, such as a 0.014 inch guidewire.

In some arrangements, an extruded three lumen catheter body is prepared in accordance with techniques known in the art. One lumen, intended as guidewire lumen 52, can have an internal diameter of at least about 0.016 inches. The wall surrounding lumen 52 can thereafter be cut down using conventional cutting or grinding equipment. Alternatively, the catheter body can be integrally molded with one lumen shorter than the other two, such as by injection molding about removable wire mandrels, and post molding cutting steps.

The distance between the distal end of lumen 52 and the proximal end of stent 18 can range from essentially zero up to an inch or more, particularly if a cover 60 is used as described infra. In one arrangement, the distance between the distal end of lumen 52 and the proximal end of stent 18 is no more than about 12 inches, and in another arrangement no more than about 0.2 inches. In some arrangements, as illustrated in FIG. 9, the distal end of lumen 52 can be about 0.08 inches from the proximal end of stent 18, and about 0.5 inches from port 15.

In some arrangements, a distal extension of the longitudinal axis of lumen 52 can be aligned to extend through the lumen 24 in temporary stent 18. In this manner, a guidewire which is threaded distally through lumen 52 can thereafter be directed through lumen 24. This design can facilitate removal and reinstallation of the guidewire while the catheter 50 is in place.

As an optional feature in accordance some arrangements, the proximal neck of one or both of the balloons 30, 32 can extend in a proximal direction to form a seal 56 around housing 54. In this manner, a cover 60 can be provided for the proximal end of lumen 24. Cover 60 can both assist in the withdrawal of the catheter from the vascular system, as well as assist in ensuring that a guidewire advanced distally through lumen 52 is guided into lumen 24. In some embodiments, the cover 60 can be provided with a plurality of perfusion ports 58 to permit continued perfusion through cover 60 and lumen 24. In some arrangements, the cover 60 can comprise a proximal extension of delivery balloon 32.

As an additional optional feature of certain arrangements, there is provided a flexible, generally cone-shaped distal tip 62 for facilitating distal advancement of the catheter 50 along a previously positioned guidewire (not illustrated). Distal tip 62 can comprise a relatively large diameter proximal portion 64 which can be an integral extension of either inflation balloon 30 or delivery balloon 32. Tip 62 can taper radially inwardly in a distal direction to a relatively narrow portion 66 having an axially-aligned guidewire and perfusion opening 68 therein.

The axial length of distal tip 62 can be varied depending upon a variety of factors such as the diameter and ridgidity of the material used. The distal tip 62 can be made from the same material as delivery balloon 32, and can be formed by axially stretching the distal end of balloon 32 with the application of heat. The proximal port diameter can be about 0.035 to 0.050 inches and the distal opening 68 in one embodiment can have a diameter of about 0.016 inches. The axial length of tip 62 can be about 0.4 inches.

To optimize perfusion through lumen 24, a plurality of ports 70 are distributed about the periphery of distal tip 62. Ports 70 can have a diameter of at least about 0.030 inches, and generally as many ports 70 (and ports 58) as possible can be provided without unduly interfering with the structural integrity of the tip 62 (or cover 60). The precise configuration of distal tip 62 can be varied considerably, while still performing the function of providing a guide for the guidewire and permitting optimum perfusion through lumen 24.

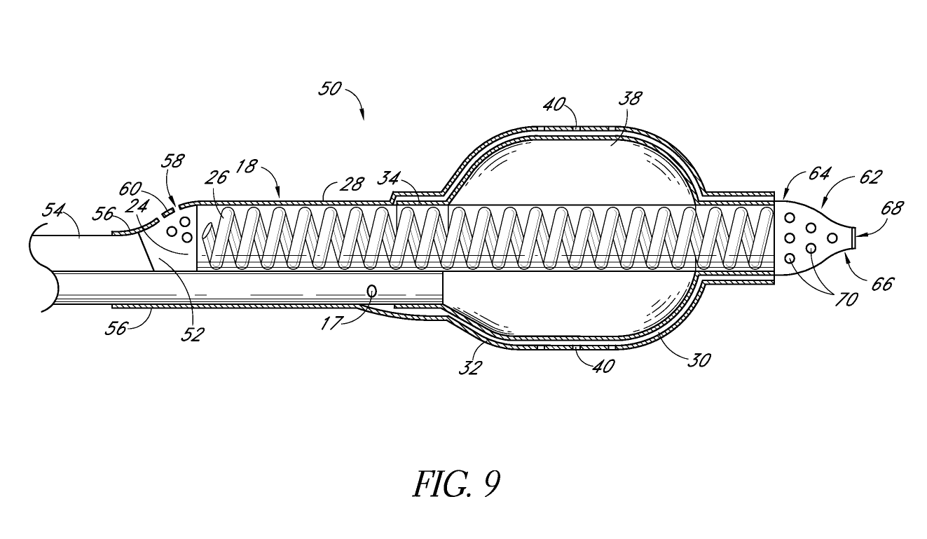

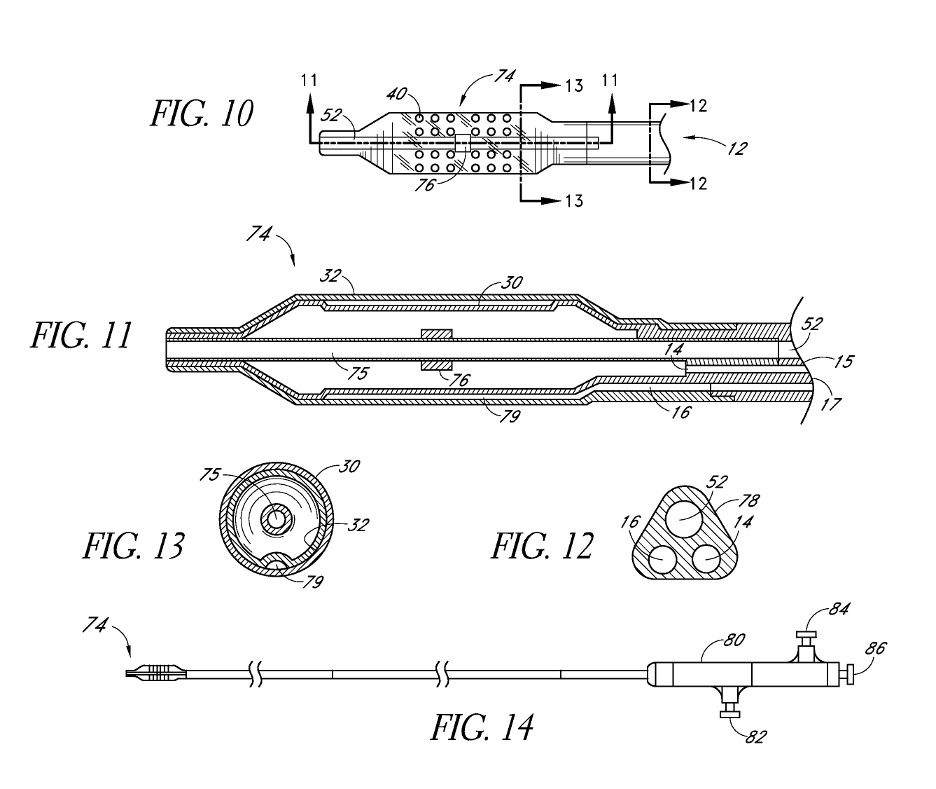

Referring to FIGS. 10-14, there is shown a nonperfusion catheter embodiment 74 which, in some embodiments, does not include a temporary stent. The non-perfusion embodiment 74 can be designed for use in percutaneous coronary transluminal angioplasty and adjunctive site specific intraluminal infusion of pharmacological agents.

The non-perfusion embodiment 74 can comprise a tubular body 12 which can include an inflation lumen 14, a drug delivery lumen 16, and a guidewire lumen 52. Two concentric balloons, an inner inflation balloon 30, and an outer delivery balloon 32 can be connected to the tubular body 12. Alternatively, the inflation balloon and delivery balloon can be disposed on opposing sides of the longitudinal axis of the body 12, such as for delivery of medication to an eccentric delivery site.

The inflation lumen 14 can be in fluid communication with the inflation balloon 30 through port 15, the delivery lumen 16 can be in fluid communication with the drug delivery balloon 32 through port 17, and the guidewire lumen 52 can be in communication with a central lumen 75 which can allow a guidewire to pass through the distal end of the catheter. A radiopaque marker 76 can be placed around the central lumen 75 in the center of the inflation balloon 32 to assist in positioning the catheter in the desired location. In some embodiments, the tubular body 12 can be an integrally formed, three lumen catheter body 78.

In the illustrated arrangement, the three lumen catheter body 78 can have a triangular cross section for a majority of the length of the tubular body 12, as illustrated in FIG. 12. The triangular shape of the tubular body 12 can provide a clearer fluoroscopy picture of the tubular body 12 within the patient, as the tubular shape reduces the cross sectional area of the tubular body 12 by up to 30%. The reduction in cross sectional area of the tubular body 12 can allow for the injection of up to 30% more dye into the guiding tube (not shown) which can provide a clearer fluoroscopy picture of the tubular body within the patient. Further, the reduction in cross sectional area of the tubular body 12 can allow for more perfusion to occur around the catheter body 12.

In the illustrated embodiment, a distal extension of the longitudinal axis of the guide wire lumen 52 can be aligned with a central lumen 75. In this manner, a guidewire which is threaded distally through lumen 52 will thereafter be directed through lumen 75. This design can facilitate removal and reinstallation of the guidewire while the catheter 74 is in place.

As illustrated in FIG. 13, the central lumen 75 can be typically concentric with both the inflation balloon 30 and delivery balloon 32 and can extend through the center of the inflation balloon 30 and exit out the distal end of the catheter. The delivery lumen 16 can extend into the catheter body and can be in fluid communication with the delivery balloon 32. As described infra, during infusion of a fluid into the delivery balloon a small luminal channel 79 can be maintained between the inflation and delivery balloons 30, 32 to enable the flow of the fluid to the delivery ports 40. The inflation lumen 14 can terminate at the proximal end of the catheter body and is therefore not shown in FIG. 13.

The inflation and delivery balloons 30, 32 can be between 2.0 cm and 6.0 cm in length. However, balloon length can be varied depending upon the requirements of a particular desired application. The deflated profile of the inflation and delivery balloons 30, 32 can be between 0.025 inches and 0.070 inches in diameter. The inflation balloon 30 and delivery balloon 32 are sealed, using a process which will be described infra, such that a portion of the distal ends and a portion of the proximal ends of the balloons are sealed together.

The delivery balloon 32 can include a series of discrete delivery ports 40 to enable the delivery of the infused liquid to the desired location. The delivery ports can be between 100 μm and 300 μm, and in other arrangements can be about 250 μm in diameter. The discrete delivery ports 40 are can be disposed radially symmetrically about the outer periphery of the delivery balloon 32 and cover the mid section of the balloon. Depending on the size of the delivery balloon 32, there can be from approximately three to fifty delivery ports in the delivery balloon 32. Alternatively, fewer delivery ports 40 can be used and disposed only on one hemisphere of the balloon or only the distal end of the balloon, depending on the desired drug delivery pattern.

In the non-perfusion embodiment, due to the relatively large diameter of the delivery ports 40 and the large number of ports 40 on the catheter, the drug can slowly drip or “weep” out of the ports 40. The large number of the large sized delivery ports 40 and the initial low pressure which is used to infuse the drug into the catheter opening can result in a very low outlet pressure at the ports 40 of the catheter tip and can therefore cause the drug to “weep” out of the ports 40 rather than exiting under a high pressure flow. The “weeping” action can cause the drug to exit the catheter tip at a site specific location, however the low pressure delivery of the drug may not be enough to penetrate the arterial wall beyond the elastic lamina layer. The delivery of the drug to the artery while maintaining the structural integrity without the penetration of the drug past the luminal wall of the artery will herein be referred to as intraluminal drug delivery, i.e., within the arterial lumen. Further, depending on the use of the catheter, i.e., for PTCA dilation, for drug delivery or for both operations, the level of inflation of the inflation balloon 30 will influence the drug delivery rate as described infra.

In another embodiment of the non-perfusion catheter, the size of the delivery ports 40 can be reduced to reduce the “weeping” effect and enable a steady flow of the drug to be delivered to the desired vascular site. In a further embodiment, the size of the delivery ports 40 can remain the same as described above and the drug delivery pressure can be increased to provide a steady flow of the drug to the desired vascular location. Generally, the total cross sectional area of all ports can be at least 300% greater and no more than 400% greater than the cross sectional area of the delivery lumen 16. In a one embodiment, the total area of the delivery ports 40 and the pressure of the fluid which is delivered to the vascular site can be both varied to achieve the desired delivery profile to the vascular site.

In some arrangements, of the non-perfusion catheter, the delivery balloon 32 can comprise a material which is inherently permeable and/or porous, without the provision of discrete delivery ports 40. For example, woven or braided filaments or fabrics can be used. For relatively low delivery rate applications, fluid permeable membranes can also be used. In certain embodiments, the balloon 32 can be selectively permeable and/or porous, for example, made porous by the application of a release agent.

Drug delivery using the non-perfusion embodiment 74 can be performed alone or in combination with a conventional PTCA procedure. When used in combination with a conventional PTCA dilation operation, the drug can be delivered before, during or after the PTCA procedure. In some arrangements, the non-perfusion embodiment 74 will be used to deliver thrombolytic agents, such as urokinase, t-PA and the like, when indicated.

When drug delivery is performed before or after conventional PTCA, the inner inflation balloon 30 can be inflated or deflated to a relatively low pressure, such as to 0.5 ATM or between about 0.4 ATM-1.5 ATM. With reference to FIG. 13, a small luminal channel 79 can be maintained between the inner inflation balloon 30 and the outer delivery balloon 32. The luminal channel 79 is typically on the order of approximately 0.01 inches in diameter when the inflation balloon 30 is inflated to a constant 0.5 ATM. Channel 79 can permit communication of the drug from delivery lumen 16 to the outer ports 40 in the delivery balloon 32 at an even and continuous rate. As the pressure applied to the drug delivery balloon 32 increases, the flow rate out of the ports 40 can increase. However, the risk of a sufficiently high pressure to perforate the vascular wall can be minimized by appropriate sizing of the channel 79 with respect to the total cross sectional area of the ports 40 as will be readily understood by one skilled in the art. Drug delivery before the PTCA dilation may be advantageous as any thrombus which is located near the area to be treated can be, but is not required to be, used for the delivery.

When the inner inflation balloon 30 is inflated to between 2 ATM and 12 ATM, the catheter can be used for dilation of a stenosis using conventional PTCA techniques. During the PTCA procedure, a drug can also be introduced into the delivery balloon 32 and delivered through the ports 40 to the specific location on the arterial wall. Even during the PTCA procedure, the resultant pressure within the delivery balloon 32 is not enough to deposit the drug into the laminal layer of the arterial wall. Drug delivery during a PTCA procedure can be advantageous to assist in treating the stenosis while the dilation is occurring. After the PTCA procedure is complete, if additional thrombus is discovered, the catheter may be used to deliver medication to the newly discovered thrombus.

Once the drug delivery and or PTCA procedure is complete and the catheter is prepared for extraction from the artery, the pressure can be first reduced at the outer delivery balloon 32 to halt continual infusion of the drug during extraction. However, the outer delivery balloon 32 may not immediately collapse. Next, the pressure in the inner inflation balloon 30 can be reduced such as by aspiration with the inflation syringe, causing the inner balloon 30 to deflate. The inner and outer balloons 30, 32 are sealed together at both axial ends, as described below, thus the reduction in diameter of the inner balloon 30 can reduce the profile of the outer balloon 32.

In some embodiments, at least a portion of the inflation balloon 30 can be connected to at least a portion of the delivery balloon 32. This structure can permit the inflation balloon to “pull” the delivery balloon with it when the inflation balloon is being aspirated to minimize the external dimensions. The connection between the inflation balloon 30 and delivery balloon 32 can be accomplished in any of a variety of techniques as will be understood by one of ordinary skill in the art.

To provide a relatively small delivery site, the inflation balloon 30 and drug delivery 32 balloon can be heat sealed together along almost the entire axial length of the balloon, leaving only a relatively small unsealed area to allow the delivery of the desired drug. To provide a relatively large delivery site, while maintaining the advantage of “pulling” the delivery balloon 32 in with the inner inflation balloon 30, only the very ends of the inflation balloon 30 and delivery balloon 32 can be sealed together. In addition, as the diameter of the delivery ports 40 increases, the percentage of the axial length of the two balloons 30, 32 that is sealed together can be increased to enable the outer delivery balloon 32 to be “pulled” in by the aspiration of the inner balloon 32. Further, as the overall pressure used to aspirate the inner balloon decreases, the percentage of the axial length of the two balloons 30, 32 that is sealed together can also be increased.

In some arrangements, about 25% of the total axial length of the inflation balloon 30 can be sealed to the delivery balloon 32 at the proximal end and about 25% of the total axial length of the inflation balloon 30 can be sealed to the delivery balloon 32 at the distal end to aid in the deflation process as described above. Desirably, the entire circumference of the distal ends of the inflation 30 and delivery balloons 32 can be sealed together. A relatively large percentage of the proximal ends of the inflation balloon 30 and delivery balloon 32 can be sealed together. The small portion of the two balloons 30, 32 on the proximal end that is not sealed together can form the very small luminal channel 79 between the inflation balloon 30 and the delivery balloon 32.

FIG. 14 illustrates the non-perfusion embodiment 74 of the catheter in communication with a fluid delivery and guidewire entry apparatus 80. An inflation port 82 can be provided for the delivery of the inflation fluid to the inflation lumen 14. A delivery port 84 can be provided for delivery of the infusion fluid to the delivery lumen 16. Port 86 can permit entry of a guidewire into the guidewire lumen 52. The guidewire entry port 86 can be positioned along the longitudinal axis of the catheter to easily align the guidewire with the guidewire lumen 52 to prevent any unnecessary bending of the guidewire during insertion into the lumen 52. The fluid delivery and guidewire entry apparatus 80 can remain outside the patient so the doctor can control the delivery of the fluid and the guidewire from outside the patient's body. In an alternate embodiment, an indeflator (not shown), which can be a syringe connected to a pressure reading device, can be attached to the inflation and delivery ports 82, 84 to monitor the pressure of the fluid which is delivered to the inflation and delivery balloons 30, 32.

The catheters incorporating various features discussed above can be manufactured in a variety of ways. Some of the preferred manufacturing techniques for catheters described herein are discussed below.

The perfusion conduit or temporary stent 18 assembly can be manufactured by winding a coil of suitable spring wire, typically having a diameter or thickness dimension in the radial direction of the finished spring of about 0.002 inches. The wire can be wound about a mandrel sufficient to produce a spring having a lumen 24 with a diameter of about 0.039 inches.

The coil can be provided with an outer sheath or coating, as has previously been discussed. In some embodiments, the tightly coiled wire can be held securely about the mandrel such as by clamping or soldering each end to the mandrel so that the coil is not permitted to unwind slightly and expand radially following release. The tightly wound coil can be thereafter inserted within a tubular sleeve, such as an extruded non-crosslinked polyethylene tubing of desired size. The spring coil can then be released from the mandrel, so that the spring can unwind slightly within the polyethylene tube to produce a tight fit.

In some embodiments, the minimum wall thickness of extruded polyethylene tubing as discussed above can be no less than about 0.002 inches. This wall thickness can be reduced by heat stretching the polyethylene tubing either prior to insertion of the spring or directly onto the pre-wound spring coil to provide a tight seal. The heat stretching step has been determined to produce a polyethylene coating on the spring coil having a wall thickness as low as about 0.001 inches. Thus, the overall diameter of the stent 18 assembly can be reduced by about 0.002 inches.

The body of the catheter can be separately produced, typically by a combination of extrusion and post-extrusion processing steps. For example, an elongate triple lumen triangular cross section catheter body can be produced by extrusion of high density polyethylene, to produce a body having a minimum wall thickness within the range of from about 0.003 to about 0.005 inches.

To minimize the overall cross sectional area of the assembled catheter, the distal portion of the tubular body 12 can be reduced in diameter and wall thickness such as by axially stretching under the influence of heat. Stretching can be accomplished by inserting, in a preferred embodiment, a 0.016 inch diameter pin in the guidewire lumen 52, and a 0.010, inch diameter pin in each of the inflation lumen 14 and drug delivery lumen 16. The distal end of the catheter body can thereafter be heat stretched nearly to the limit before breaking. The result of the stretching can reduce the cross-section of the triangular catheter body, from base to apex, from about 0.039 inches in the unstretched condition to about 0.025 inches following heat stretching.

The transition zone between the unstretched catheter body 12 and the distal axially stretched portion can occur within about 0.01 inches proximally of the proximal end of the temporary stent 18 in the assembled catheter. It has been determined that the decrease in structural strength of the heat stretched catheter body does not appear to adversely impact the integrity of the assembled catheter, in some embodiments of the designs disclosed herein.

The inflation balloon 30 and drug delivery balloon can be manufactured in any of a variety of manners which are now conventional in the art, such as free-blowing polyethylene, polyethylene terephthalate, nylon, polyester, or any of a variety of other medical grade polymers known for this use. Generally, the interior inflation balloon 30 can be produced by blowing relatively long sections of cross-linked polyethylene within a mold to control the outside diameter. The use of cross-linked polyethylene can facilitate heat sealing to the coil, which can be coated with non-crosslinked polyethylene.

The sections of inflation balloon material can thereafter be heat stretched at the proximal and distal necks of a balloon down to a thickness of about 0.001 inches and a diameter which relatively closely fits the portion of the catheter body to which it is to be sealed. The appropriate length can be cut, depending upon the desired length of the balloon and balloon necks in the finished catheter.

The proximal neck can be heat sealed around the catheter body 12 and the temporary stent 18, as illustrated in FIGS. 5 and 9. In general, the length of the proximal and distal neck which is secured to the catheter body can be within the range of from about 0.05 inches to about 0.1 inch, except in an embodiment such as illustrated in FIG. 9, in which the proximal and distal balloon necks can be as long as necessary to accomplish their functions as a proximal cover or distal tip. The distal end of the inflation balloon 30 can thereafter be heat sealed around the distal end of the temporary stent 18.

The outer balloon can thereafter be assembled in a similar manner, following “necking down” of the axial ends of the balloon by axial stretching under the application of heat. In an embodiment utilizing cross-linked polyethylene for the outer delivery balloon, the delivery balloon can be secured to the axial ends of the inflation balloon through the use of a UV-curable adhesive, due to the difficulty in thermally bonding cross-linked polyethylene to cross-linked polyethylene.

However, it is to be understood that the material utilized for the outer delivery “balloon” can be varied and the term “balloon” as used in the context of the delivery balloon is intended to be only generally descriptive of this structure. For example, in addition to perforated balloons, a wide variety of materials not conventionally used for true balloons may also be used. Woven or braided fibers such as dacron, or fluid permeable membranes can be used for the outer delivery balloon, as has been discussed.

In some arrangements, the cross-sectional configuration of the temporary stent 18 can change from substantially circular at the distal end thereof to substantially rectangular or square at the proximal end thereof. This configuration can be accomplished by winding the spring coil around a mandrel having a square cross-sectional portion, a transition portion, and a round cross-sectional portion. The transition portion on the resulting spring is located in the assembled catheter at about the line 4-4 on FIG. 7. This can allow the temporary stent portion 18 to retain the same internal cross-sectional area, while reducing the maximum width of the assembled catheter.

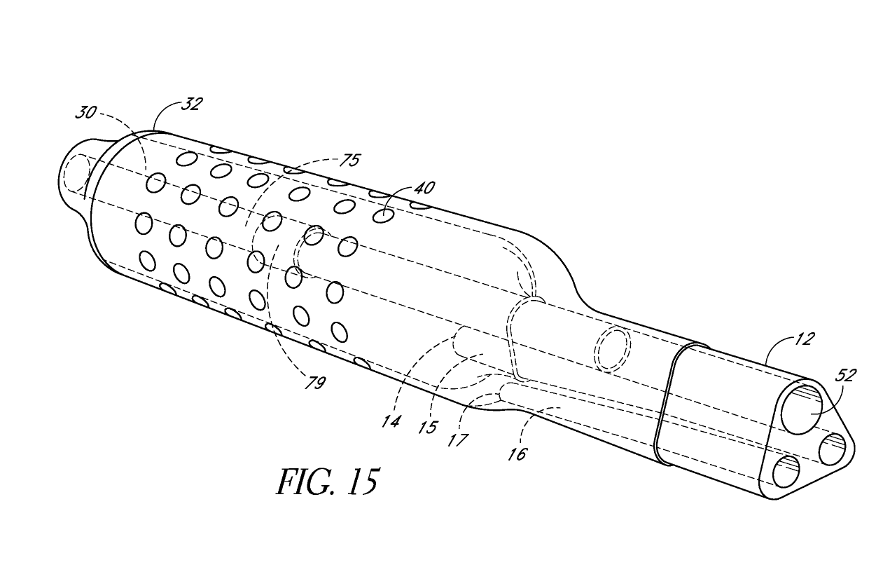

In the non-perfusion embodiment 74, the distal end of the catheter body 12 can be cut away to separately expose each of the three lumen as illustrated in FIG. 15. First, a small portion of the catheter body can be cut away to expose the drug delivery lumen 16. Next, a larger length can be cut away to expose the inflation lumen 14. Finally, an additional portion can be cut away to expose the guidewire lumen 52. The central lumen 75 can abut the guidewire lumen and the two lumen can be joined together using an adhesive or any other suitable bonding process. A radioopaque marker 76 can be positioned in the center of the catheter 74 concentric to the central lumen 75.

A long steel mandrel can be inserted into each of the inflation lumen 14, delivery lumen 16, and the guidewire lumen 52 which extends through the central lumen 75, the mandrels extending along the entire length of the catheter body 12. The steel mandrels can be provided to keep the lumen from sealing closed during the balloon assembly procedure. The inflation balloon 30 can be placed over the central lumen 75 and the inflation lumen 14. The inflation balloon 30 can then be bonded to the central lumen 75 and the inflation lumen 14 at the proximal end and to the central lumen 75 at the distal end. The inflation balloon 30 can be bonded to the inflation lumen 14 and the central lumen 75 using any of a variety of bonding techniques known to those skilled in the art, such as solvent bonding, thermal adhesive bonding, or by heat sealing. In some arrangements, the inflation balloon 30 can be heat sealed to the inflation lumen 14 and the central lumen 75.

The delivery balloon 32 can be bonded to the catheter body 12 by any of a variety of bonding techniques such as solvent bonding, thermal adhesive bonding or by heat sealing depending on the type of balloon material used. In some arrangements, crosslinked polyethylene balloons can be used, therefore the inflation 30 and delivery balloons 32 can be heat sealed together as follows. The wire mandrel can be removed from the central lumen 75 and guidewire lumen 52 and a 0.01 inch diameter teflon rod can be placed in the central lumen 75 to inhibit or prevent that the central lumen 75 from sealing closed during the assembly process.

The delivery balloon 32 can be positioned at the proximal end of the catheter 74 to cover the inflation balloon 30 and the delivery lumen 16. To create the luminal channel 79, a teflon rod of a diameter which can be the same as the desired diameter of the luminal channel 79 can be placed between the inflation balloon 30 and the delivery balloon 32 at the proximal end of the two balloons 30, 32. A teflon capture tube (not shown) can be positioned over the delivery balloon 32 and can cover the portion of the proximal end of the delivery balloon 32 which is to be sealed to the inflation balloon 30. In some embodiments, the teflon capture tube can be a generally tubular body which can have approximately the same diameter as the inflated diameter of the inflation balloon 30 and can be made of teflon. The inflation balloon 30 can be inflated to a pressure which is sufficient to force the delivery balloon 32 against the wall of the teflon capture tube. In some embodiments, the inflation balloon 30 can be inflated to about 30-50 psi. The capture tube can be heated by any of a number of heating means such as electric coils or a furnace to a temperature which is sufficient to bond the two balloons 30, 32 together. For example, the crosslinked polyethylene balloons can be heated to a temperature of about 300 degrees Fahrenheit which can cause both balloons to seal together. The teflon capture tube can then be cooled to a temperature below the melting temperature of the two balloons 30, 32. The inflation balloon 30 can be deflated and the catheter can be removed from the capture tube. The teflon rod used to create the luminal channel 79 can be removed.

To seal the distal end of the delivery balloon 32 to the inflation balloon 30, the delivery balloon can be positioned at the distal end of the catheter 74 and can substantially or completely cover the inflation balloon 30. The teflon capture tube (not shown) can be positioned over the delivery balloon 32 and can cover the portion of the distal end of the delivery balloon 32 which is to be sealed to the inflation balloon 30. The inflation balloon 30 can be inflated to force the delivery balloon 32 against the wall of the teflon capture tube. The inflation balloon 30 can be inflated to about 30-50 psi. As above, the capture tube can be heated by any of a number of heating means such as electric coils or a furnace to a temperature which is sufficient to bond the two balloons 30, 32 together. For example, the crosslinked polyethylene balloons can be heated to a temperature of about 300 degrees Fahrenheit which can cause both balloons to seal together. The teflon capture tube can then be cooled to a temperature below the melting temperature of the two balloons 30, 32. The inflation balloon 30 can be deflated and the catheter removed from the capture tube. The teflon rod can be removed through the distal end of the central lumen 75. The steel mandrels can be removed from the inflation lumen 14 and the delivery lumen 16 through the proximal end of the catheter body 12.

In some arrangements, a site is identified in a body lumen where it is desired to deliver an amount of a medication or other gas or fluid. For example, thrombolytic or restenosis inhibiting drugs can be introduced directly to the affected wall following dilation. Alternatively, anticoagulants, plaque softening agents or other drugs may desirably be delivered directly to the site of a thrombosis or other vascular anomaly.

A conventional angioplasty guidewire can be percutaneously transluminally inserted and advanced to the desired treatment site. Guidewires suitable for this purpose are commercially available, having a variety of diameters such as 0.014 inches.

The distal end 22 of temporary stent 18 can be threaded over the proximal end of the guidewire once the guidewire has been positioned within the desired delivery site. The catheter 10 can be thereafter advanced along the guidewire in the manner of conventional “over-the-wire” balloon angioplasty catheters. A conventional guidewire having an exterior diameter of about 0.014 inches can have a cross-sectional area of about 0.000154 inches, and a temporary stent 18 having an interior diameter of about 0.039 inches can have an interior cross-sectional area of about 0.001194 inches. The cross-sectional area of the interior lumen 24 of stent 18, which remains available for perfusion once a guidewire is in place, can therefore be about 0.00104 square inches.

The catheter 10 can be advanced through the vascular system, along the guidewire, until the drug delivery balloon 40 is disposed adjacent the desired delivery site. Thereafter, a suitable inflation fluid such as a radiopaque solution can be introduced by way of lumen 14 into the inflation balloon 30 to press the delivery balloon 32 against the vascular wall. Although described herein in its drug delivery capacity, the catheter may alternatively be used to perform dilation, as has previously been described.

Once the drug delivery balloon 40 is positioned adjacent the vascular wall, medication can be infused by way of lumen 16 in tubular body 12 and expelled through effluent ports 40 directly against the vascular wall. Medication can be introduced under gravity feed alone, or by way of a positive pressure pump, as desired by the clinician in view of such factors as drug viscosity, toxicity and desired delivery time.

In this manner, drugs can be permitted to be absorbed directly into the affected site, with a minimal amount of drug escaping into generalized circulation. The rate of drug delivery can be somewhat limited by the rate of absorption by the vascular wall, and delivery rates on the order of about 30 ml per hour to about 20 ml per minute can be used. Certain medications can be optimally delivered at much lower rates, such as 1 ml per day or lower. However, these rates can be modified significantly, depending upon the drug, and the extent to which “overflow” fluid is permitted to escape into the circulatory system.

In the drug delivery application, in some embodiments, delivery of a sufficient amount of drug may require an extended period of time. Perfusion past the delivery balloon by way of temporary stent 18 can minimize the adverse impact on circulation due to the indwelling drug delivery catheter. Following infusion of the predetermined volume of drug, and optionally following a further “rinse” with a sufficient volume of N-saline to expel substantially all of the drug from the residual volume of lumen 16 and space between drug delivery balloon 32 and inflation balloon 30, the inflation balloon 30 can be deflated and the catheter can be withdrawn. Alternatively, the catheter 10 can be introduced by way of an introduction sheath having a lumen with a large enough diameter to accommodate catheter 10.

During the foregoing procedures, the guidewire (not illustrated) can either be removed or can be left in place, as will be understood by one of skill in the art. In general, cardiologists prefer to leave the guidewire in place so that the catheter may be withdrawn and replaced, or other catheters may be inserted.

In a modified method, the catheter 10 can be utilized as a temporary stent for an observation period following percutaneous transluminal coronary angioplasty, atherectomy, laser ablation or any of a variety of other interventional catheter techniques and procedures. In some embodiments of the apparatus, the drug delivery balloon 32 can be deleted entirely, and the tubular body 12 can optionally be provided with only a single fluid lumen extending therethrough to provide communication with the interior of inflation balloon 30.

Following removal of an interventional therapeutic catheter, such as an angioplasty, atherectomy or laser ablation catheter, the temporary stent catheter 10 can be inserted along the guidewire or through an introduction sheath and disposed with the inflation balloon 30 at the previously treated site. Inflation balloon 30 can be inflated to the desired diameter to resist reocclusion during a post-procedure period. Such observation periods may vary depending upon the circumstances of the patient and the cardiologist, but generally range from about 30 minutes to about 24 hours. During this time, perfusion across the inflation balloon 30 can be permitted by way of temporary stent 18.

As has been previously described, the relative cross-sectional area of the lumen 24, even with an indwelling guidewire, permits a significant degree of perfusion to occur. In addition, the longitudinal axis of lumen 24 can be generally concentric with or parallel to the longitudinal axis of the artery or vein in which the indwelling temporary stent is disposed. In this manner, the interruption of direction of blood flow can be minimized, thereby reducing the likelihood of damaging blood cells and introducing undesired turbulence.

In some arrangements, portions of the inflation balloon 30 and/or the drug deliver balloon 32 of the above-described catheter arrangements can carry, for example, a therapeutic that does not readily dissolve in an aqueous solution, such as, for example, paclitaxel. Paclitaxel is a lipophylic agent and does not readily dissolve in aqueous solution. Paclitaxel can be dissolved in ethanol or any other organic solvent that does not form micelles. A portion of the balloon 30, 32 can be dipped or otherwise coated in the solution and subsequently dried. Those of skill in the art will recognize that in other embodiments the therapeutic agent can be carried by the balloon 30, 32 in other manners, such as, for example, embedding the material, otherwise depositing the material on the surface of the balloon, and/or dispersing the material within the balloon material.

The coated balloon catheter can be used to dilate stenotic arterial lesions using standard intervention procedures. The balloon 30, 32 can be inflated to dilate the artery at the site of the lesion. While inflated, a bolus of release agent can be injected into the outer porous balloon 32 to release paclitaxel from the coated portions of the balloon 30, 32 and facilitate its transport into the aortic wall. Solvents such as ethanol can be used to release paclitaxel and dissolve it in solution. Alternatively or in addition, contrast medium including commercially available Visipaque 320, Omnipaque, or Magnevist can be used to improve the solubility of Paclitaxel.

The release of the therapeutic agent can be stopped or greatly reduced by injecting saline into the outer balloon 32 to inhibit the dissolution process. An advantage of the above-described arrangement is that the release of the therapeutic agent can be controlled by a second agent (release agent) that is injected through the catheter. The dose of therapeutic agent released will be dependent on the potency of the release agent and the duration of application. In some embodiments, this can be an improvement over existing methods of drug delivery via drug-coated surfaces, in which the delivery rate is predetermined by the composition and properties of the coating. The above-described catheter and method can provide for improved and individualized dosing of the drug during the procedure. Furthermore, injection of excessive amount of release agent will not overdose the patient. Surplus amount of release agent can be washed into the blood stream without impacting the release of the therapeutic agent.

The above-described method and apparatus of placing a therapeutic agent on the surface of a drug delivery system and subsequently control the release the therapeutic agent with a release agent can be extended to other combinations of therapeutic drugs and release agent. For example, Lipophilic therapeutic agent do not readily dissolve in aqueous solutions such as blood. Organic solvents can be used to release lipophilic drugs from the surface of the delivery system.

In some arrangements, the therapeutic agents can be placed in a degradable polymeric carrier that is coated onto the drug delivery device. For example poly amino-ester is a known biodegradable polymer for drug delivery. The poly amino ester can be formulated such that it degrades rapidly at acidic pH. The therapeutic agent can be added to the poly amino ester and the drug delivery device can be coated with the solution. At physiological pH (pH 7.2), the coating can be fairly stable, retaining the therapeutic agent during the insertion and placement of the drug delivery system. Once the coated surface of the drug delivery device is positioned at the target site, a release agent of low pH (pH 5.0-6.5) can be injected to accelerate the degradation of the polymer and release the therapeutic agent. Using a pH-sensitive biocompatible drug carrier is only one example of biodegradable carriers that can be used to retain the drug. Other biodegradable carriers can be used with degradation rates dependent on other the physical properties of the solution besides pH. For example, carriers can be considered with a degradation rate dependent on the temperature or ionic concentration of the solution. The release agent can be designed accordingly to change the physical or chemical properties of the solution to increase the rate of degradation and such the release of the therapeutic agent.

In some embodiments, the therapeutic agent can be be chemically bonded to the surface using reversible chemical bonds. For example, tannins including catechin can be added to the coating to retain the therapeutic agent using weak hydrogen bonds. The release agent can include substances with a higher affinity to tannin. Large proteins such as collagen are known to have a high affinity to tannins. Collagen would compete with and replace the therapeutic agent in the hydrogen bonds, effectively releasing it into solution. Those of skill of the art will recognize that there are many chemical reactions that could be used to initially bond the therapeutic agent to a surface and subsequently release the agent by a second reaction that is initiated by administering a release agent. The release rate can be controlled by the concentration of the release agent and the duration of application.

The drug delivery device and method that utilizes the release agent described above is not limited to the catheter arrangements described in U.S. Pat. No. 5,295,962 and FIGS. 5-15 described above. Those of skill in the art will recognize the principles of utilization of the therapeutic agent and release agent described above can be extended and applied to other devices the delivery of drugs into diseased locations in the body such as blood vessels, organs, and tumors. In such modified arrangements, the drug delivery device need not include the dual balloon arrangement described above but can use a single balloon and/or another type of expandable or moveable member. In such arrangements, the therapeutic agent can be retained on the surface of the device in contact or in vicinity to the treatment site and the therapeutic agent can be released from the surface by the administration of a second agent through the delivery device. The surface of the device can comprise a balloon or other moveable element. However, it is also anticipated that the surface can be a fixed or semi-fixed member.

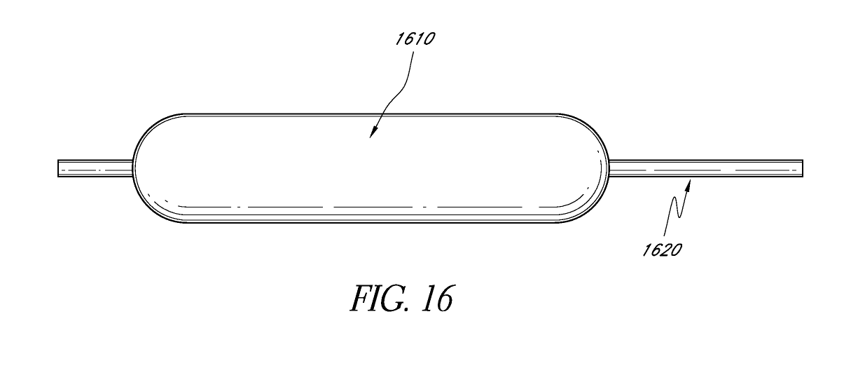

With reference now to FIG. 16, there is shown another embodiment of a drug delivery catheter. The catheter consists of an inflatable balloon 1610 that is mounted onto a catheter. The catheter can contain a lumen 1620 to inflate the balloon 1610. The general design of the balloon catheter can be similar to that of existing balloon catheters for angioplasty. They are referred to in the literature as angioplasty catheters, PTCA, and PTA catheters. However, as explained below, in this embodiment, the loading and release of a drug from the balloon 1610 is new and provides certain advantages.

Drugs that may be considered include anti-thromogenic agents such as Heparin, magnesium sulfate, or anti-proliferation drugs such as Paclitaxel and Rapamycin, or photodynamic agents, or drugs to prevent extra-cellular matrix degeneration such as Catechin and doxycycline. While Paclitaxol generally has limited solubility in aqueous solutions, hydrophilic forms of Paclitaxol, for example, those that might be chelated to binding groups such as polyethylene glyoocl or polysaccharides, are considered in this technology.

The balloon 1610 can be made from a semi-elastic or elastic polymers or elastomer that is sensitive to a solvent, i.e. so that the balloon 1610 can swell when exposed to a solvent. Balloon materials include, but are not limited to, latex, vinyl, silicone, polyurethane, and nylon. Solvents include, but are not limited to, acetone ethyl acetate, alcohol, and ethanol.

The agent can be dissolved in the solvent in preparation for loading the balloon 1610 with the agent. The concentration of agent can be chosen such that the agent has a therapeutic effect when loaded and subsequently released from the balloon 1610. For example 2 mg/ml of Paclitaxel can be dissolved in 100% ethyl acetate or 5 mg/ml Catechin can be dissolved in 100% acetone. The concentration of the solvent in the solution depends on the resistance of the balloon material to the solvent. For example, low-durometer polyurethane has a low resistance to acetone whereas nylon 6-6 has a high resistance to acetone. Balloon material composed of multiple polymers, for example, balloons that are co-extruded or alternately dip cast such that a low durometer polymer is contained over a high durometer polymer can be used.

The balloon 1610 can be immerged in the solution containing the solvent and the agent. The solvent can cause the balloon 1610 to swell and to facilitate the absorption of the agent into the balloon wall. In some embodiments, the balloon 1610 can be immerged either in a collapsed state, or an inflated state at low pressure, or an inflated state at high pressure. Inflating the balloon 1610 can expose the surface of the balloon 1610 more uniformly to the solvent. The balloon 1610 can be inflated to a high pressure to stretch the balloon material and increase the permeability of the balloon 1610. The balloon 1610 can be immerged in solution for a few minutes, for example 1-5 minutes. The balloon 1610 can be subsequently dried to flash off the solvent. The process of emerging the balloon 1610 in the solvent and drying can be repeated one or several times to increase the concentration of the agent in the balloon wall.

After the solvent is removed from the balloon 1610, the agents can remain trapped on and in the micro-structure of the balloon 1610. In some embodiments, when the balloon 1610 is inserted into a blood vessel, the balloon 1610 can be configured so that the agent will not readily escape from the balloon wall. In some embodiments, only small amounts of agent will be released in the blood stream. When the balloon 1610 is inflated in the target vessel against the vessel wall, the balloon material can be stretched and the permeability/microporosity of its microstructure can be increased. In some embodiments, the agent can be rapidly released from the balloon 1610. At the same time, the endothelial layer on the internal surface of the blood vessel can be stretched. It is well know that the endothelial cells do not stretch with the extra-cellular matrix. Stretching of the arterial wall can create gaps between the endothelial cells that act as channels for the agent to enter the extra-cellular matrix. Effectively, balloon angioplasty can temporarily increase the permeability of the endothelium for rapid drug delivery. In some embodiments, the balloon 1610 can be inflated beyond the nominal diameter of the target vessel. This is in contrast to other proposed drug delivery balloon systems that are intended to merely make contact with or conform to the inner wall of the artery for drug delivery. This is a noted advantage of the above-described embodiment.

When the balloon 1610 is inflated, the balloon material can be exposed to high stresses. Angioplasty balloon are typically inflated to 2-12 atm. Balloon materials are therefore typically made from material with high tensile strength such as PE or nylon. In PTCA, rigid balloons that do not stretch and retain there shape when inflated can be used. This allows the clinician to pre-select the exact balloon diameter best suited for a particular blood vessel. For drug delivery, a semi-elastic and elastic balloon material can be used. Furthermore, the tensile strength of the balloon material can be compromised when exposed to a solvent. For example, polyurethane is known to crack after long exposure to a solvent. Thus, there exist competing design constraints for the construction of a drug-delivery balloon catheter as described here. Further, because different polymers will have different molecular structures, the micro-porosities of these materials can vary.

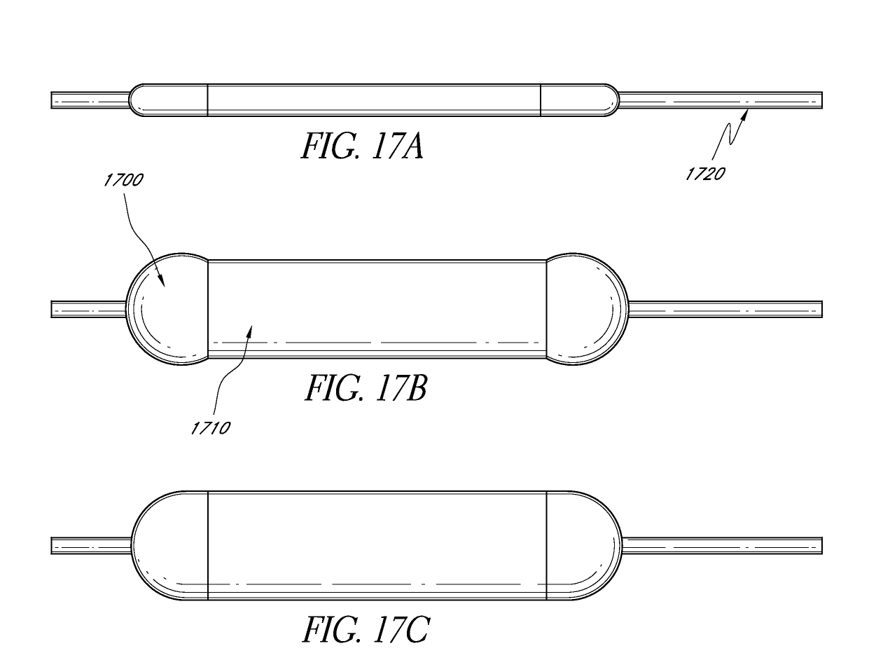

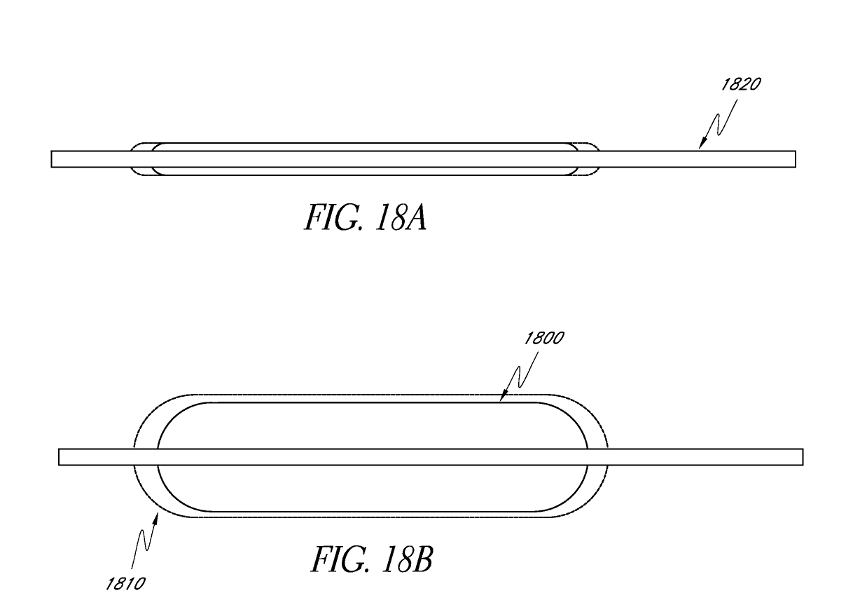

The above-described design constraints can be overcome by designing a balloon catheter with two co-axial balloons as shown in FIGS. 17A-18B. The outer balloon 1710 can be a semi-elastic or elastic balloon containing the agent. The inner balloon 1700 can be a standard angioplasty balloon. The inner balloon 1700 can be inflated during angioplasty. The outer balloon 1710 containing the agent can be expanded by the inner balloon 1700. The relaxed diameter of the outer balloon 1710 can be less than that of the inner balloon 1700, such as when the inner balloon 1700 is rigid or semi-rigid. The outer balloon 1710 can be stretched for rapid drug release when the inner balloon 1700 is inflated to its nominal size.

To deflate the outer balloon 1710 for catheter retraction, the shoulders of the inner balloon 1700 and the outer balloon 1710 can be bonded together as described by Crocker (U.S. Pat. Nos. 5,295,962 and 5,569,184) and as described above. Alternatively, the outer balloon 1710 can be connected to a separate inflation lumen to deflate the balloon separately.

In some embodiments the outer balloon can be constructed from a highly elastic material such as latex with stretch ratios over 100%. The collapsed profile of the outer balloon can be similar to the profile of the catheter shaft. Upon deflation of the inner balloon, the outer balloon can be configured to collapse back to its original diameter, requiring no or little additional deflation.

In some embodiments, balloon can have an inner layer that can provide the mechanical strength and an outer layer than can contain the therapeutic agent.

The method of loading and releasing a therapeutic agent from a layer of polymer or elastomer can be incorporated into a variety of drug delivery systems. For example, a stent graft may be constructed with a graft that contains a therapeutics agent. In some embodiments, the therapeutic agent can be embedded in the wall of the graft. Upon deployment, the stent can expand the graft such that the graft is positioned against the blood vessel wall near the mural thrombus. The therapeutic agent can then be released into the mural thrombus to facilitate reduction of enzymatic degradation of protein and promote cross-linking of protein in the extracellular matrix.

Some embodiments of the apparatuses and methods disclosed herein can be configured to deliver the agent into the smooth muscle cells within the aortic wall. For example, paclitaxel generally enters the cell in order to down-regulate its proliferation. Paclitaxel does not easily pass through the cell membrane. It is proposed to use PEI (polyethylene imide). PEI has a high affinity to paclitacel and can act as a carrier to cross the cell membrane. Alternatively, in some embodiments, other chelating agents may be used, such as, but not limited to, ethylene diamine tetraacetic acid (“EDTA”).

Another embodiment relates to the surface tension of balloon material. A high surface tension of the material can repel absorption of aqueous solutions. For that reason an organic solvent can be used to transport the therapeutic agent into the ePTFE matrix. When the balloon is inserted into the blood vessel, it is exposed to the blood stream. Because blood is an aqueous solution, it generally cannot penetrate into the ePTFE matrix. Only the agent on the surface of the balloon is potentially removed. The bulk of the agent stays within the porous structure of the balloon. Blood will generally only penetrate into the matrix and extract the agent when the surface tension is reduced. This can be done by injecting an organic solvent at the time of balloon inflation. Alternatively, the surface tension can be reduced by applying physical pressure to the surface of the balloon. When the balloon is pressed against the vessel wall, pressure is exerted onto the balloon surface breaking the surface, tension and allowing blood serum to penetrate into the matrix and extract the agent. Therefore, expansion of the balloon beyond the diameter of the blood vessel is an important aspect of this invention.

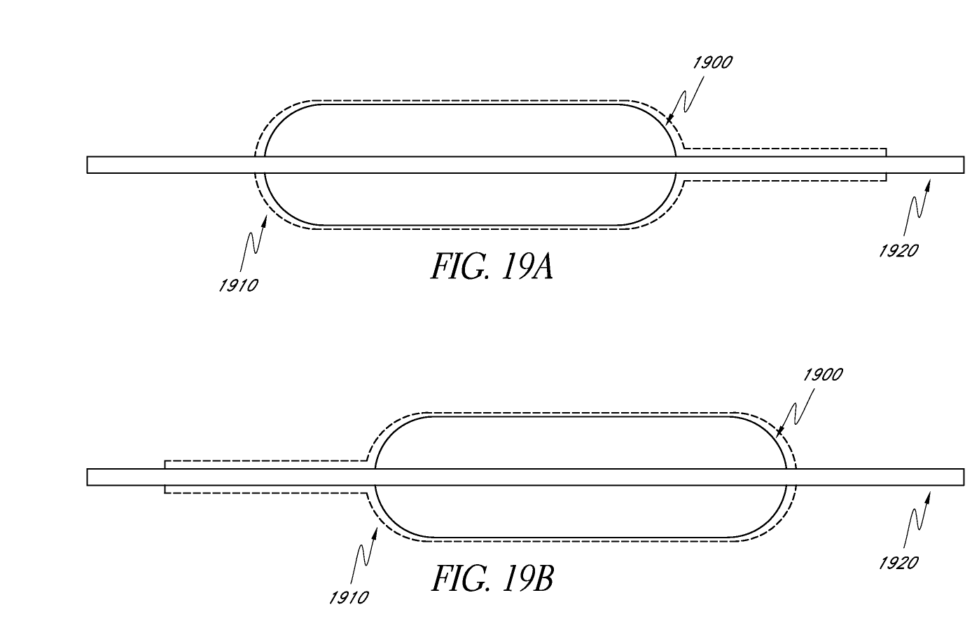

It may be advantageous to treat longer lesions of a diseased blood vessel in case of diffuse atherosclerotic disease. Long lesions may require the use of multiple drug-delivery balloons. In some embodiments, the outer balloon 1910 can be substantially longer than the inner balloon 1900, spanning most of all the length of the lesion, as illustrated in FIGS. 19A-19B. The inner balloon 1900 can move axially inside the outer balloon 1910, allowing for the inflation of individual sections of the outer balloon 1910. Thus, long lesion can be treated with one balloon catheter.

In some embodiments, the balloons or other apparatuses described herein can comprise a latex material. In some embodiments, the following method can be used to load in the latex balloon with a therapeutic agent. However, the method is not limited to latex and can be applied to other elastic materials such as silicone and polyurethane. Polyethylene glycol (PEG) also can be added to the latex emulsion. Various molecular weights of PEG can be used. In some embodiments, a lower molecular weight PEG can be used to improve the dispersion of PEG in the latex emulsion. In some embodiments, PEG with a molecular weight between approximately 100 and approximately 1000, or between approximately 200 and 400 can be used. The concentration of PEG in the emulsion can be between approximately 0.05% and approximately 5%, or between approximately 0.5% and approximately 2%.

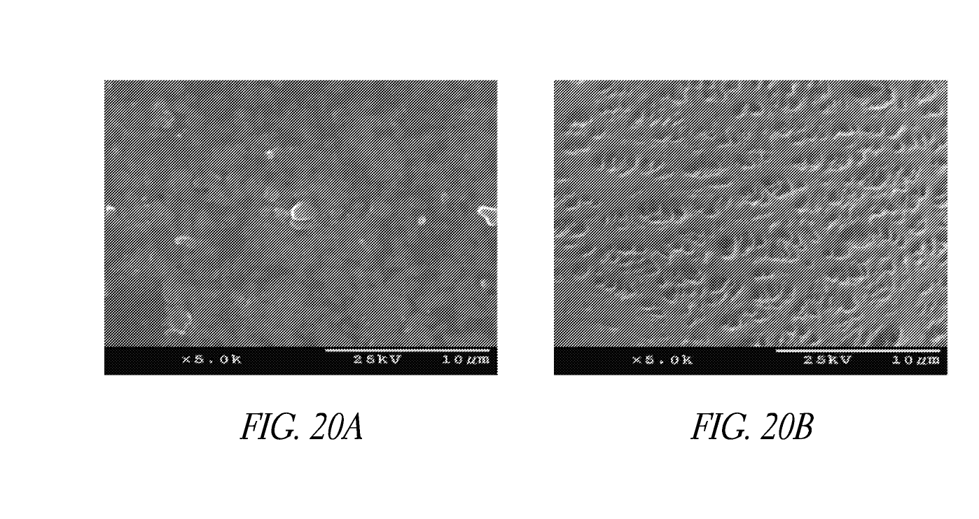

In some embodiments, the PEG can interfere with the cross-linking, thereby locally disrupting the micro structure of the latex. The PEG can be removed from the cured balloon with an organic solvent. FIG. 20A shows an SEM image at 5.0 k magnification of a latex surface prepared with 1% PEG having molecular weight of between approximately 380 and approximately 420. As seen in FIG. 20A, the surface of the latex can be generally smooth with the indication of some granulation. FIG. 20B shows an SEM image at 5.0 k magnification of the surface of the latex shown in FIG. 20A, stretched to about 400% of its original dimensions. Micropores can be created where PEG interfered with the cross-linking of the latex. When the stretched latex balloon is immersed in a solution containing an organic solvent and a therapeutic agent, the solution can penetrate into the micro pores. The solution can then be evaporated, leaving the agent in the pores. The latex can then be collapsed, trapping the agent in the microstructure. The balloon can be inserted into the blood stream in a collapsed state. In some embodiments, only small amounts of agents will elute from the balloon in the collapsed state. Once the balloon is inflated and contacts the wall of the blood vessel, serum can enter the micro pores and transport the agent into the vessel wall.

In some embodiments, the agent can be physically trapped in the micropores of the elastic balloon. In some embodiments, no chemical bonding of the agent to the balloon, which can alter the properties of the agent, is required. Also, in some embodiments, no chemical bonding has to be overcome to release the agent from the balloon. In some embodiments, the therapeutic agent can be delivered with other agents that increase the dissolution of the agent in serum, or the transport of the therapeutic agent into the wall, or increase the permeability of the extracellular matrix or cell membranes, or increase the residence time of the agent in the vessel wall. For example chelating agents such as PEI and EDTA can increase the dissolution of paclitaxel, increase the affinity to cell and the extracellular matrix, and increase cell permeability. Any of these additional agents can be added to the solution containing the therapeutic agent and loaded into the microstructure of the balloon. Different agents can be loaded into the balloon sequentially using separate solutions for each agent.

It would be understood to one of ordinary skill in the art of medical balloon manufacturing that various balloon materials and agents can be used to create a porous matrix. For example, in some embodiments, salt microparticles can be added to the emulsion, dissolved, and removed after curing. Alternatively, the cured material can be exposed to a strong organic solvent such as acetone to break down the molecular structure at the surface of the balloon. In some embodiments, micropores can be created that substantially enlarge when the balloon material is stretched from its collapsed state to its inflated state.

In some embodiments, catechin can be delivered into the wall of the blood vessel. The catechin can contain at least EGCG and ECG. In some embodiments, the catechin can have between approximately 20% and approximately 60% EGCG, and between approximately 5% and approximately 30% ECG. In some embodiments, the inflation time of the balloon can be between approximately 10 seconds and approximately 60 minutes or more, or between approximately 1 min and approximately 15 minutes. This can be different from long-term application of agents eluting from a device.

In addition, catechins can be applied to local vessel injuries to promote healing, restore normal function of the endothelium, reduce thrombosis, stabilize the extra-cellular matrix via cross-linking and inhibition of enzymatic degradation, reduce inflammation, and inhibit smooth muscle cell proliferation. Injuries to the vessel wall may be caused by atherosclerosis, vulnerable plaque, angioplasty, stent placement, atherectomy, surgical anestomosis, and endovascular devices. It will be obvious to one of ordinary skill in the art that catechins can be used for treating a wide range of local vessel injuries or diseases. Some embodiments of the present disclosure relate to a short-term treatment of a local lesion in the blood vessel.

In other embodiments, paclitaxel and a chelating agents such as PEI or EDTA can be loaded and delivered with the microporous balloon. The chelating agent can enhance the transport of paclitaxel to and into the targeted smooth muscle cells.

The above-described method and apparatus of placing a therapeutic agent on the surface of a drug delivery system and subsequently controlling the release the therapeutic agent with a release agent can be extended to other combinations of therapeutic drugs and release agent. For example, Lipophilic therapeutic agent may not readily dissolve in aqueous solutions such as blood. Organic solvents can be used to release lipophilic drugs from the surface of the delivery system.

In certain embodiments, vascular ePTFE grafts can be manufactured by extruding PTFE tubing, sintering the extruded material to obtain mechanical strength and mechanically stretching and expanding the material to obtain the desired final geometrical and mechanical specification. Improvements to the surface biocompatibility contemplated by prior art typically include the application of a surface coating to the final ePTFE graft.

In some embodiments, an ePTFE graft can be loaded with Catechin, for example EpiGalloCatechin Gallate (EGCG), to decrease its thrombogenicity. The molecular structure of catechins is shown in FIG. 1. Other flavenoids and catechin compounds may also be considered that are known to have a therapeutic effect. To introduce the agent into the ePTFE structure, the agent is dissolved in acetone. Other organic solvent systems such as alcohol and acetate may also be considered. The solvents should be able to penetrate the ePTFE without damaging its structure. ePTFE is highly resistant to organic solvents and therefore well suited as a drug carrier. The graft is submerged in the acetone solution containing the agent. Alternatively, in some embodiments, only the lumen of the ePTFE graft can be filled with acetone solution containing. A pressure gradient can be created across the graft by pressurizing the graft or applying vacuum to the outside of the graft to facilitate penetration of the acetone solution. For example, EGCG has a low molecular weight (less than 1000) and is readily transported into the porous matrix of the ePTFE graft by the acetone. The graft can then be dried to flash off the acetone while permitting the EGCG to remain in the matrix.

The concentration of EGCG in acetone can be between about 0.01% and about 10%, and in some embodiments between about 0.1% and about 1%. The desired concentration in a particular application can be dependent on the desired release rate and desired anti-thromogenic surface properties. The graft can be submerged in the acetone solution for 30 seconds to several hours, preferably between 1 minute and 10 minutes. The acetone solution can be applied multiple times to increase the concentration of EGCG in the graft.

In some embodiments, the ePTFE graft can be a tubular endovascular graft that can be supported by a support structure, such as the endovascular grafts described in U.S. Pat. No. 6,733,523, entitled “Implantable Vascular graft,” filed on Jun. 26, 2001, the entirety of which is herein incorporated by reference. Those of skill of the art will recognize that various embodiments and/or aspects thereof of the grafts disclosed in the '523 patent can be combined with the various features described herein to produce additional embodiments of an endovascular graft having certain features and advantages according the present invention.

EGCG also exhibits anti-hyperplastic properties. When surgical grafts are connected to blood vessels, the blood vessel is exposed to increased stresses at the anastomosis. This is particularly true for veins in A-V shunt procedures. The mechanical stresses cause smooth muscle cell proliferation and migration into the vessel lumen. The migrating smooth muscle cells effectively reduce the size of the vessel lumen and can completely obstruct the lumen. This process is referred to as intimal hyperplasia. It is a common failure mode of small diameter grafts. In the initial sequence of the process, Matrix Metalloproteinase (MMP) is released from the smooth muscle cells to break down the collagen matrix and path the way for cell migration. EGCG suppresses the activity of MMPs and hence reduce cell migration into the lumen. See U.S. Pat. No. 6,214,868 for details on the mechanism of EGCG. In one embodiment of the invention, EGCG is released from the ePTFE graft into the adjacent tissue at the anastomosis site. Grafts are typically sutures or stapled to the blood vessel. The pressure created by the sutures or staples forces body fluid into the porous structure of the ePTFE. EGCG dissolves readily in aqueous solutions such as blood and is rapidly transported into the tissue. EGCG also has a high affinity to protein, specifically collagen, preventing a wash-out into the blood stream.

Another aspect of the present invention disclosure to the high surface tension of ePTFE. The ePTFE material repels aqueous solutions. For that reason, an organic solvent can be needed in some embodiments to transport the therapeutic agent into the ePTFE matrix. When the ePTFE graft is implanted, it is exposed to blood and saline. Because these fluids are aqueous solutions, they generally cannot penetrate into the ePTFE matrix. Only the agent on the surface of the ePTFE graft is readily removed. The bulk of the agent stays within the porous structure of the graft. Blood can only penetrate into the matrix and extract the agent when the surface tension is reduced. This could be done by adding a solvent. Alternatively, the surface tension can be reduced by applying physical pressure to the surface. As mentioned earlier, sutures and staples used to perform the anastomosis press the tissue against the graft and break the surface tension. The surface tension can also be reduced by blood elements contacting the surface of the ePTFE. Proteins are known to reduce surface tension. When platelets adhere to the surface of the ePTFE, they also enhance the release of catechin, which in return inhibit platelet aggregation.

In some embodiments, the porosity of the graft can be varied along the graft to optimize drug release. Along the inner layer of the graft, a small pore size may be desirable to minimize platelet adhesion. At the anastomosis sites, a large pore size may be advantageous to maximize the loading of EGCG for the prevention of hyperplasia. The concentration of EGCG in the graft may also be increased at the anastomosis sites by multiple applications of the acetone solution to the ends of the graft.

In some embodiments, the ePTFE graft can have of several layers. In some embodiments, only the inner blood-contacting layer can be treated with EGCG. The un-treated outer layer can promote blood coagulation and adhesion to the blood vessel.

It is understood that many other therapeutic agents that can be dissolved in an organic solvent can be applied to the ePTFE graft. They include, but are not limited to, Heparin, Paclitaxel, Rapamycin, and doxycycline.

Additionally, the apparatuses and methods disclosed herein for tissue stabilization are not limited to applications involving aneurysms or dissections. The apparatuses and methods disclosed herein can be used for treating other diseased conditions of the vascular system, and other suitable vessels. For example, without limitation, rupture of the vaso vasorum of the aorta can create an intramural hematoma, which is a thrombus within the layers of the aorta. A hematoma may develop into a dissection. A therapeutic agent can also be injected or delivered in the hematoma using any of the apparatuses or methods disclosed herein to stabilize the surrounding tissue against enzymatic degeneration. Therefore, the term “mural thrombus” as used herein should be interpreted broadly and is meant to refer to any thrombus adjacent to a targeted extracellular matrix layer, including a thrombus associated with a dissection.

Although the inventions have been disclosed in the context of preferred embodiments and examples, it will be understood by those skilled in the art that the present disclosure extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It can be also contemplated that various combinations or subcombinations of the specific features and aspects of the embodiments can be made and still fall within the scope of the invention. Accordingly, it should be understood that various features and aspects of the disclosed embodiments can be combined with or substituted for one another in order to form varying modes of the disclosed invention. Thus, it can be intended that the scope of the present disclosure herein disclosed should not be limited by the particular disclosed embodiments described above.

What is claimed is:

1. A method for stabilizing an extracellular matrix in a wall of a blood vessel comprising:
advancing a delivery system to a treatment site positioned near a mural thrombus that covers at least a portion of the wall of the blood vessel;
advancing a delivery portion of the delivery device into the mural thrombus;
delivering a therapeutic agent through the delivery portion into the mural thrombus; and
allowing the agent to transport from the mural thrombus into the extracellular matrix of the vessel wall by diffusion to facilitate reduction of enzymatic degradation of protein in the extracellular matrix by the action of the agent.

2. The method of claim 1, wherein the agent cross-links with proteins in the extracellular matrix and protects the protein against enzymatic degradation.

3. The method of claim 1, wherein the agent is a bioflavonoid selected from the group consisting of: proanthocyanidin, catechin, epicatechin, epigallo catechin, epicatechin gallate, epigallocatechin gallate, quercetin, tannic acid, and any combination thereof.

4. The method of claim 3, wherein the bioflavonoid is EGCG.

5. The method of claim 1, wherein the agent is in a solution.

6. The method of claim 5, wherein the solution containing the therapeutic agent has a pH less than 7.4.

7. The method of claim 5, wherein the solution has a pH close to the isoelectric point of collagen or elastin.

8. The method of claim 1, wherein the extracellular matrix layer is located in an aortic aneurysm or aortic dissection.

9. The method of claim 1, wherein delivering the therapeutic agent comprises a catheter.

10. The method of claim 9, wherein the catheter comprises at least one ejection port perpendicular to the axis of the catheter.

11. The method of claim 1, wherein a concentration of the therapeutic agent delivered into the mural thrombus is substantially higher than the concentration of the therapeutic agent in the extra-cellular matrix.

12. The method of claim 1, wherein a concentration of the therapeutic agent delivered into the mural thrombus is between approximately 2.0% and approximately 10.0%.

13. A method for stabilizing an extracellular matrix layer in the vascular system of a body comprising:
positioning a portion of a vascular catheter adjacent to or within a mural thrombus positioned adjacent to the extracellular matrix layer of a target region of the vascular system; and
delivering a therapeutic agent in solution to the mural thrombus using the vascular catheter; and
allowing the therapeutic agent to be transported to the extracellular matrix layer through the mural thrombus to promote the cross-linking protein in the extracellular matrix layer, thereby stabilizing the extracellular matrix.

14. The method of claim 13, wherein the therapeutic agent is a bioflavonoid.

15. The method of claim 14, wherein the bioflavonoid forms at least one hydrogen bond with protein in the extracellular matrix layer.

16. The method of claim 13, wherein the solution contains a keotropic agent.

17. The method of claim 16, wherein the keotropic agent is Ca(OH)₂.

18. The method of claim 13, wherein the therapeutic agent is delivered to the mural thrombus using a stent graft.

19. The method of claim 18, wherein the stent graft comprises ePTFE.

20. The method of claim 13, wherein the therapeutic agent is delivered to the mural thrombus using at least one expandable balloon configured to expand against the mural thrombus.

21. A method of claim 13, wherein the therapeutic agent is an anti-inflammatory, anti-platelet agent or a matrix-metalloproteinase inhibitor.

22. A method of claim 13, wherein the solution contains at least 2% catechin.

23. A method of claim 13, wherein the solution contains at least 10% catechin.

24. A method of claim 13, wherein the solution contains an organic solvent.

25. A method of claim 24, wherein the organic solvent is acetone, alcohol, ethyl acetate, methanol, or methyl acetate.

26. A method of claim 24, wherein the solution contains at least 10% of an organic solvent.

27. A catheter system for the delivery of a therapeutic agent into a wall of a blood vessel, comprising:
a delivery catheter, wherein the delivery catheter houses a therapeutic agent configured to promote the cross-linking of protein; and
a delivery portion of the delivery catheter, wherein the delivery portion is configured to deliver the therapeutic agent from the delivery catheter into a mural thrombus.

28. The catheter system of claim 27, wherein the delivery catheter has a first proximal end and a second distal end, wherein the first and second ends are connected by a delivery lumen and wherein the second distal end comprises an atraumatic tip.

29. The catheter system of claim 28, further comprising at least one ejection port disposed on a side of the catheter between the first and second ends, wherein the ejection port is oriented perpendicular to the axis of the catheter.

30. The catheter system of claim 28, wherein the first proximal end further comprises a reservoir.

31. The catheter system of claim 28, wherein the atraumatic tip comprises a radiopaque marker.

32. The catheter system of claim 28, wherein the lumen is configured to house a guidewire.

33. The catheter system of claim 28, wherein the atraumatic tip is articulated.
